SEQUENCE LISTING (12 sequences)

SEQ ID NO: 1 - DNA, 21 bp, Artificial Sequence
  GAPDH sense
ggagcgagatccctccaaaat

SEQ ID NO: 2 - DNA, 23 bp, Artificial Sequence
  GAPDH antisense
ggctgttgtcatacttctcatgg

SEQ ID NO: 3 - DNA, 23 bp, Artificial Sequence
  Caspase 3 sense
tacctcttatgaggagaaacggt

SEQ ID NO: 4 - DNA, 22 bp, Artificial Sequence
  Caspase 3 antisense
aggaaagtccaggtctagcttg

SEQ ID NO: 5 - DNA, 23 bp, Artificial Sequence
  Caspase 6 sense
catgctgggaagatactgttgat

SEQ ID NO: 6 - DNA, 23 bp, Artificial Sequence
  Caspase 6 antisense
gcccgagactaacaaaagactct

SEQ ID NO: 7 - DNA, 21 bp, Artificial Sequence
  Caspase 8 sense
gggagcctcttgcaggataaa

SEQ ID NO: 8 - DNA, 21 bp, Artificial Sequence
  Caspase 8 antisense
gaatggggcatagctcaccac

SEQ ID NO: 9 - DNA, 23 bp, Artificial Sequence
  Caspase 9 sense
tgtcttggaatgcactgtatctc

SEQ ID NO: 10 - DNA, 22 bp, Artificial Sequence
  Caspase 9 antisense
cccagtaaggctgtaaatgctc

SEQ ID NO: 11 - DNA, 21 bp, Artificial Sequence
  Caspase 12 sense
tggagctggtaacccagtagg

SEQ ID NO: 12 - DNA, 23 bp, Artificial Sequence
  Caspase 12 antisense
tggtacctttgccttggagtatt

(19) United States
(12) Patent Application Publication
(10) Pub. No.: US 2022/0211869 A1
(43) Pub. Date: Jul. 7, 2022

(54) PHENYLALANINE FUNCTIONALISED MESOPOROUS SILICA NANOPARTICLES AS DRUG-FREE NANOTHERAPEUTICS OR BIOACTIVE NANOCARRIER FOR ANTI-CANCER APPLICATIONS

(71) Applicant: Nanyang Technological University, Singapore (SG)

(72) Inventors: Chor Yong TAY, Singapore (SG); Zhuoran WU, Singapore (SG)

(21) Appl. No.: 17/535,062

(22) Filed: Nov. 24, 2021

(30) Foreign Application Priority Data
Nov. 27, 2020 (SG) 10202011840Q

(51) Int. Cl. A61K 47/69; A61K 47/54; A61P 35/00

(52) U.S. Cl. CPC A61K 47/6929; A61P 35/00; A61K 47/542; A61K 47/545

(57) ABSTRACT

The present invention provides an amino acid-functionalized nanoparticle, comprising: a nanoparticle having a dimension in the range of units of nanometers to 100 nanometers, and having an external surface; and a plurality of amino acid molecules conjugated to the external surface of the nanoparticle, the amino acid molecules having a chemical property that can induce a cancer cell to ingest the functionalized nanoparticle; a method of producing such a nanoparticle; and uses of the nanoparticle.

### CROSS-REFERENCE TO RELATED APPLICATIONS

The present application claims priority to Singapore Application No. SG 10202011840Q filed with the Intellectual Property Office of Singapore on Nov. 27, 2020 and entitled “PHENYLALANINE FUNCTIONALISED MESOPOROUS SILICA NANOPARTICLES AS DRUG-FREE NANOTHERAPEUTICS OR BIOACTIVE NANOCARRIER FOR ANTI-CANCER APPLICATIONS,” which is incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4373-17200_SP103391USBD ST25; created on Feb. 15, 2022; and having a files size of 3 KB.

The information in the Sequence Listing is incorporated herein in its entirety for all purposes.

### FIELD OF THE INVENTION

The present invention relates to functionalised mesoporous silica nanoparticles and/or other ROS-generating nanoparticles for anti-cancer applications, methods of producing the nanoparticles, and uses thereof.

### BACKGROUND OF THE INVENTION

The classical nanomedicine paradigm relies heavily on the concept of exploiting biocompatible, and sometimes, multifunctional engineered nanomaterials (ENMs) as delivery vehicles to carry chemotherapeutic agents for anti-cancer applications. Due to the nano-scale dimensions and ability to fine tune their surface chemistries, biocompatible ENMs are well suited to be the agent of choice for (i) encapsulating and protecting the payload (drug) from pre-mature degradation and release; (ii) facilitating passage of the drug into hard to access tissue and cancer microenvironment; (iii) controlled release of the drugs and (iv) passive and/ or active targeting of the drugs into the cancer cells [Hare, J. I., et al., Adv Drug Deliv Rev 2017, 108: 25-38]. Despite the advancements in cancer-centric nanomedicine, developing a targeted treatment with appreciable effectiveness, low cost, and minimal toxic effects are longstanding cumbersome issues. One possible solution is to devise a bio-active nanomaterial that would enable us to eliminate the drug component in nanomedicine while retaining the selectivity towards cancer cells. Conceivably, realization of a safe and low-cost bio-ENMs with innate cancer killing capability will pave the way for better treatment options and affordable health care for cancer patients.

The concept of “drug free nanotherapeutics” was conceived in 2014 (Tay, C. Y, et al., Adv Funct Mater 2014, 24 (38): 5936-5955; Tay, C. Y., et al., Nanomedicine-Uk 2014, 9 (14): 2075-2077), and experimentally demonstrated where it was shown that inorganic ENMs such as silica nanoparticles, titanium dioxide nanoparticles and hydroxyapatite nanoparticles are able to disrupt microtubule polymerization in human squamous cell carcinoma, TR 146, to limit cancer cell migration (Tay, C. Y., et al., Nano Lett 2014, 14 (1): 83-88). Moreover, use of such ENMs could exploit the deficiency of p53 in DLD-1 and SW480 colorectal cell lines to elicit a preferential killing using ROS generating ZnO nanoparticles (Setyawati, M. I., et al., Biomaterials 2013, 34 (38): 10133-42). While findings from these studies suggest that pristine ENM can be employed as anti-cancer agents, issues such as uncontrollable ROS production, non-specific cytotoxicity and off-target effects are technological “choke-points” that have yet to be resolved.

Cancer cells require a constant exogenous supply or increased de novo synthesis of amino acids to support their biomass and growth (Tsun, Z. Y. and Possemato, R., Semin Cell Dev Biol 2015, 43: 22-32). The exquisite dependency of cancer cells for amino acids is well-established in studies showing that restricting amino-acids availability could profoundly limit tumor growth, and induce cancer cell death (Vucetic, M. et al., J., Front Oncol 2017, 7: 319; Lopez-Lazaro, M., Oncoscience 2015, 2(10): 857-66). Along the same vein, it was also recently demonstrated that leucine plays an indispensable role in conferring resistance to tamoxifen in estrogen receptor-positive breast cancers (Saito, Y. et al., Nature 2019, 569(7755): 275-279). Given the crucial role amino acids play in cancer cell metabolism and tumorigenesis, strategies to deprive cancer cells of exogenous sources of amino acids such as fasting and protein restriction have emerged (FIG. 1A) (Vucetic, M. et al., Front Oncol 2017, 7: 319; Lopez-Lazaro, M., Oncoscience 2015, 2(10): 857-66; Maddocks, O. D. K. et al., Nature 2017, 544(7650): 372-376). However, limiting dietary intake of amino acids may not be suitable for patients who are at risk of malnutrition or cachexia, where the reduced levels of nutrients may further aggravate the condition. Together with the noncompliance in patients to the strict dietary requirement, the clinical adoption of a nutrient-based approach to curb cancer growth by limiting amino acids intake is significantly curtailed.

There is a need for improved drug-free nanotherapeutic treatments to target cancer cells to be treated.

### SUMMARY OF THE INVENTION

The amino acid addiction displayed by cancer cells has inspired us to devise a trojan horse-like strategy: to replete the cancer cells with apoptosis-inducing nano porous amino acid mimics (Nano-PAAM), without recourse to the incorporation of pharmaceutical agents nor application of external stimuli, causing the cancer cells to self-destruct. A small library consisting of 9 essential amino-acid (EAA) based Nano-PAAM (30 nm) were synthesized to examine its anti-cancer effects. A mesoporous silica nanoparticles (MSN) core was chosen as SiO₂ is a Generally Recognized as Safe (GRAS) material, biocompatible and its size and surface chemistries can be easily tailored (Lehmen, S. E. et al., Environ Sci Nano 2016, 3(1): 56-66). Furthermore, we had previously shown that the ROS inducing capability of MSN could be tailored by controlling the particle porosity, which can potentially be exploited to induce oxidative stress-mediated cell death in cancer cells (Tay, C. Y. et al., ACS Nano 2017, 11(3): 2764-2772). Among the panel of Nano-PAAM screened, L-phenylalanine functionalized Nano-PAAM (Nano-pPAAM) exhibited the greatest potency (˜80% kill rate) against MDA-MB-231 triple-negative human breast cancer cells. Importantly, we showed that the cytotoxic potential of Nano-pPAAM in the 2D monolayer model was highly specific towards cancerous cells (i.e., MDA-MB 231, MCF-7, MKN, and 11-4). It was extremely well-tolerated by non-cancerous cells such as NCM460, HDF, and HaCaT (viability>90%). The selective killing of cancer cells was achieved in part through the targeting of SLC7A5, L-type amino acid transporter (LAT-1), that is overexpressed in cancer cells to meet its exogenous amino acid demand. Further structure-activity relationship analysis revealed that both particle size and porosity are critical determinants of Nano-pPAAM anti-cancer efficacy. Mechanistically, it was found that Nano-pPAAM can co-activate both the extrinsic and intrinsic apoptotic pathways in MDA-MB-231. As a proof-of-concept, the antitumoral properties of Nano-pPAAM was further validated using an MDA-MB 231 xenograft in vivo mice model.

The working principle of the said invention is premised on (i) the intrinsically high demand of cancer cells for amino acids (AAs) to fuel their metabolism and (ii) their susceptibility to oxidative stress-induced cell death.

According to a first aspect of the invention, there is provided an amino acid-functionalized nanoparticle, comprising:

i) a nanoparticle having a dimension in the range of units of nanometers to 100 nanometers, and having an external surface; and

ii) a plurality of amino acid molecules conjugated to the external surface of the nanoparticle, the amino acid molecules having a chemical property that can induce a cancer cell to ingest the functionalized nanoparticle;

wherein said nanoparticle is a mesoporous silica nanoparticle and/or other ROS-generating nanoparticle; and

wherein said amino acid molecules are selected from the group consisting of histidine, isoleucine, leucine, lysine, methionine, phenylalanine, threonine, tryptophan, and valine.

Preferably, said amino acids are selected from the group consisting of tryptophan, isoleucine, methionine and L-phenylalanine.

In some embodiments, the amino acid is L-phenylalanine.

In some embodiments, the nanoparticle has a dimension in the range of 10-150 nm, preferably about 30 nm.

In some embodiments, the nanoparticle is a mesoporous silica nanoparticle and has mesopore structures in the range of about 1 nm to about 5 nm in size.

In some embodiments, the biological effect is ROS-induced cancer cell apoptosis.

In some embodiments, the cancer cell overexpresses an L-type amino acid transporter 1 (LAT1) compared to a normal cell.

In some embodiments, the cancer cell is selected from the group comprising breast cancer, gastric cancer and skin cancer.

According to another aspect of the invention there is provided a method of production of an amino acid-functionalized nanoparticle of the invention, comprising:

a) Mix and dissolve an essential amino acid, ethyl(dimethylaminopropyl) carbodiimide (EDC), and N-Hydroxy succinimide (NHS) in PBS buffer;

b) Form a suspension of NH₂-functionalized mesoporous silicon nanoparticles or other NH₂-functionalized ROS-generating nanoparticles in PBS buffer;

c) Mix the suspension from b) with the solution from a) at room temperature; and

d) Extract the final product.

In some embodiments, the method of production further comprises freeze-drying the amino acid-functionalized nanoparticles product for storage.

In some embodiments of the method of production, the nanoparticle has a dimension in the range of 10-80 nm, preferably about 30 nm.

In some embodiments of the method of production, the final product is an amino acid-functionalized mesoporous silica nanoparticle.

In some embodiments of the method of production, the mesoporous silica nanoparticle has mesopore structures in the range of about 2 nm to about 3 nm in size.

In some embodiments of the method of production, the essential amino acid is selected from the group consisting of Trp, Ile, Met and Phe.

In some embodiments of the method of production, the essential amino acid is L-phenylalanine.

According to another aspect of the invention there is provided a pharmaceutical composition comprising at least one amino acid-functionalized nanoparticle of any aspect of the invention and an acceptable pharmaceutical vehicle for the treatment of cancer in a subject.

In some embodiments of the pharmaceutical composition, the cancer comprises cancer cells that overexpress an L-type amino acid transporter 1 (LAT1) compared to a normal cell.

In some embodiments, the cancer is selected from the group consisting of breast cancer, gastric cancer and skin cancer.

According to another aspect of the invention there is provided a method of treatment comprising administering to a subject in need of such treatment an effective amount of an amino acid-functionalized nanoparticle of any aspect of the invention.

In some embodiments, the subject has cancer.

In some embodiments, the cancer comprises cancer cells that overexpress an L-type amino acid transporter 1 (LAT1) compared to a normal cell.

In some embodiments, the cancer is selected from the group consisting of breast cancer, gastric cancer and skin cancer.

### BRIEF DESCRIPTION OF THE FIGURES

FIGS. 1A-B shows a schematic illustrating the working principle of (1A) conventional nutrient deprivation and (1B) the proposed nanoparticles mediated approach to kill cancer cells. Illustration created with BioRender.

FIGS. 2A-D shows (2A) Synthesis scheme of EAA based Nano-PAAMs employed in this study. (2B) Representative TEM image of Nano-PAAM. Scale bar=50 nm. (2C) FTIR spectra of the various nanoparticle variants. The feature peaks in NH₂-MSN and Nano-PAAM are highlighted by arrows with their values indicated correspondingly in the graph. (2D) in vitro anti-cancer activity of the different Nano-PAAM variants was examined using the highly invasive M DA-MB-231 human breast cancer cells. NH₂-functionalized MSN serves as the experimental control group. Viability values are presented in the form of colored heat map with its color scale ranging from medium grey (100%) to light grey (50%) to dark grey (0%).

FIG. 3 shows the Nano-pPAAM prepared using the acid reflux method, but not the calcination method, could possess the anti-cancer property on MB231 cells.

FIG. 4 shows the equivalent concentration of EAA conjugated to the MSN determined at 205 nm. Data are presented as mean±standard deviation.

FIGS. 5A-D shows representative transmission electron microscope (TEM) images of Nano-pPPAM with diameter of (5A) 80 nm, (5B) 170 nm, (5C) and 260 nm, as well as (5D) Nano-pSiNP (30 nm). Scale bar=100 nm.

FIGS. 6A-D shows anti-cancer killing mechanism and selective cancer targeting of Nano-pPAAM is mediated via LAT-1. (6A) in vitro assessment of the cancer killing activity of Nano-pPAAM as a function of particle size in MDA-MB-231 cells. (6B) Measured cell viability of MDA-MB-231 cells treated with Nano-pPAAM with or without BCH (10 mM) treatment. (6C) Comparative analysis of LAT-1 expression in selected cell lines. n=40/group. (6D) Dose-dependent analysis of normal and cancerous cell lines treated with either Nano-pPAAM, or NH₂-MSN, or cisplatin. Data are presented as mean±standard deviation. * denotes significant difference between the respective experimental group and control group. p<0.05. Viability values are presented in the form of colored heat map with its color scale ranging from mid grey (100%) to light grey (50%) to dark grey (0%).

FIGS. 7A-D shows the cellular uptake of FITC-Nano-pPAAM (30 nm) at 500 μg/ml by MDA-MB-231 cells. Cells were post-treated with trypan blue to quench the signal from the surface bound FITC-Nano-pPAAM. (7A) Measured mean FITC signals/cells for each of the experimental groups. (n=10/group) Data are presented as mean±standard deviation. * denotes a significant difference between sample group and control group at p<0.05. Representative fluorescence images of cells treated with FITC-Nano-pPAAM (arrow heads) at (B) 37° C., (7B) co-treated with NaN₃ (10 mM) and (C) 4° C. Cell nuclei were counter-stained with Hoechst dye (not seen in greyscale). Scale bar=50 μm

FIG. 8 shows the blockage of endocytosis by low temperature significantly reduced the anticancer potency of Nano-pPAAM in MDA-MB231 cells. Nano-pPAAM pre-exposed cells were cultured at either 37° C. or 4° C. for 6 h followed by removing the particles from the cell culture medium and allowing the cells to grow for another 18 h, prior to viability examination by alamarBlue. Data are presented as Mean±SD. * states significant difference between sample group and control group. p<0.05.

FIG. 9 shows the cell uptake of Nano-pPAAM and NH₂-MSN by MDA-MB-231 cells with or without BCH (10 mM). Internalized Nano-pPAAM and NH₂-MSN were measured by ICP-MS. Data are presented as mean±standard deviation. * denotes a significant difference between sample group and control group. p<0.05.

FIG. 10 shows the representative immunofluorescence images panel normal and cancerous cells counter-stained for LAT-1 (middle row) and nucleus (top row). Scale bar=100 μm.

FIGS. 11A-D shows intrinsic ROS inducing capability of Nano-pPAAM is a critical feature needed to exert its anticancer activity. (11A) Representative fluorescence images of control, NH₂-MSN (500 μg/ml) and Nano-pPAAM (500 μg/ml) treated (8 h) MDA-MB-231 cells labelled with CELLROX Orange Reagent (middle column). Scale=50 μm. (11B) Cell viability measurement of MDA-MB-231 cells treated with Nano-pPAAM only or co-treated by Nano-pPAAM and NAC. (11C) Anticancer efficiency of Nano-pPAAM/-SiNP in MDA-MB-231 cells. (11D) ROS measurements of Nano-pPAAM/-SiNP treated cells after 8 h of treatment. n=20. TBHP (100 μM) exposure was employed as a positive control. Data are presented as mean±standard deviation. * denotes a significant difference between the respective experimental group and control group. # denotes a significant difference between Nano-pPAAM and Nano-SiNP treated group. p<0.05.

FIGS. 12A-E shows nano-pPAAM induces apoptosis in MDA-MB-231 cells via dual activation of intrinsic and extrinsic apoptotic pathways. (12A) Immunocytochemical staining images and (12B) corresponding flow cytometry analysis of MDA-MB-231 cells treated with Nano-pPAAM (500 μg/ml) for 8 h and untreated cells counter-labeled with FTIC-Annexin V (white) and PI (grey). Scale=50 μm. (12C) mRNA transcript expression of several apoptotic caspases in Nano-pPAAM treated MDA-MB-231 cells for 8 h with or without NAC treatment. (12D) Flow cytometric analysis of the cleaved caspase 8 level in Nano-pPAAM treated or untreated MDA-MB-231 cells. (12E) Measured cell viability values of Nano-pPAAM treated cells with or without the addition of caspase 8 inhibitor. Data are presented as mean±standard deviation. * denotes a significant difference between sample group and control group. p<0.05.

FIGS. 13A-C shows in vitro anti-tumor properties of Nano-PAAM. (13A) Schematic illustrating the use of micropatterned 3D agarose gel to generate uniformly-sized MDA-MB-231 cancer spheroids. Scale bar=100 μm. Time-dependent MDA-MB-231 spheroid (13B) size and (13C) cell viability measurement of Nano-pPAAM (500 μg/ml) and NH₂-MSN (500 μg/ml) treated samples. Data are presented as mean±standard deviation. * denotes a significant difference between sample group and control group. p<0.05.

FIGS. 14A-F shows in vivo anti-tumor properties and fate of Nano-pPAAM. (14A) Tumor volume changes as a function of time post administration of PBS (control), NH₂-MSN (3 mg/ml) and Nano-pPAAM (3 mg/ml). n=5 per group. Right panel: Representative images of excised tumors retrieved from the various experimental groups 14 days post treatment. Scale bar=1 cm. (14B) Changes to the whole-body weight of mice of the various experimental groups. (14C) ICP-MS analysis of the amount of Si in the excised tissues 14 days after Nano-pPAAM or NH₂-MSN treatment. (14D) Hematoxylin and eosin staining of the various tissue and tumor section harvested from the MDA-MB-231 bearing xenograft mice 14 days post treatment. Scale bar=50 μm. (14E) Representative fluorescence images of MDA-MB-231 tumor section counterstained with PI (grey) and TUNEL (white) treated with PBS (control), NH₂-MSN (3 mg/ml) and Nano-pPAAM (3 mg/ml). Scale bar=50 μm. (14F) Quantification of apoptotic cells in the tumor section as determined by TUNEL assay. Data are presented as mean±standard deviation. * denotes a significant difference between compared sample groups. p<0.05.

FIGS. 15A-B shows the cell viability measurement of (15A) Nano-pPAAM and (15B) L-phenylalanine treated cells after 24 hours of treatment. The concentration of the L-phenylalanine solution was adjusted to an equivalent concentration of L-phenylalanine conjugated onto the MSN, assuming a conjugation efficiency of 100%.

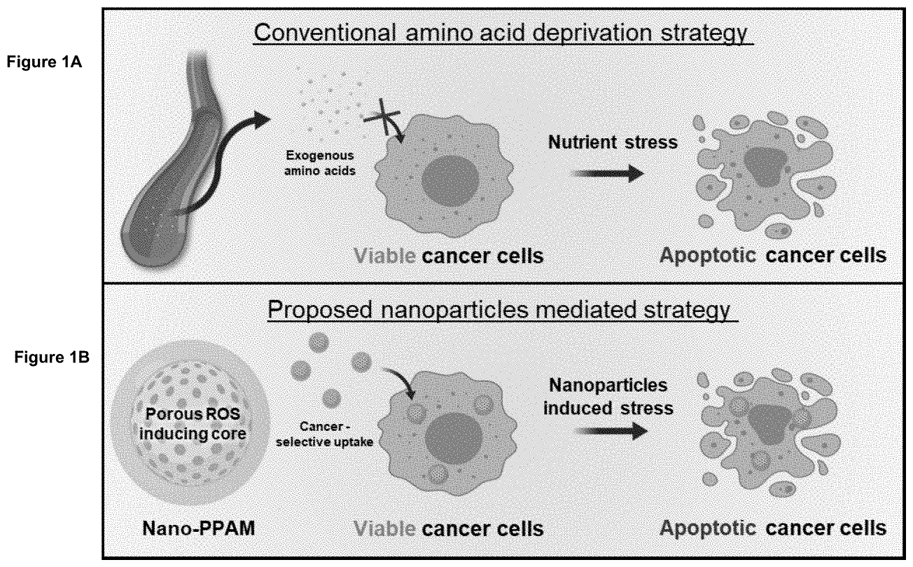

### DETAILED DESCRIPTION

A class of functionalized ROS-generating nanoparticles useful in medical treatments is described. The nanoparticles have an attached essential amino acid that is selected on the basis that a cell to be treated ingests as a consequence of the presence of the amino acid. The nanoparticles are carried into the cell where they cause ROS-induced cell death, such as cancer cell death.

### Definitions

Certain terms employed in the specification, examples and appended claims are collected here for convenience.

The term “subject” is herein defined as vertebrate, particularly mammal, more particularly human. For purposes of research, the subject may particularly be at least one animal model, e.g., a mouse, rat and the like. In particular, for treatment or prophylaxis of flavivirus infection and/or flavivirus-linked diseases, the subject may be a human.

The term “treatment”, as used in the context of the invention refers to ameliorating, therapeutic or curative treatment.

The term “comprising” is herein defined to be that where the various components, ingredients, or steps, can be conjointly employed in practicing the present invention. Accordingly, the term “comprising” encompasses the more restrictive terms “consisting essentially of” and “consisting of.”

A person skilled in the art will appreciate that the present invention may be practiced without undue experimentation according to the methods given herein. The methods, techniques and chemicals are as described in the references given or from protocols in standard biotechnology and molecular biology text books.

### EXAMPLES

Standard molecular biology techniques known in the art and not specifically described were generally followed as described in Green and Sambrook, Molecular Cloning: A Laboratory Manual, Cold Spring Harbour Laboratory, New York (2012).

### Example 1

### Methods

### Materials and Chemicals

Cetyltrimethylammonium bromide (CTAB, >99%, BioXtra), (3-Aminopropyl)-triethoxysilane (APTES, >99%), Tetraethyl orthosilicate (TEOS, 98%, reagent grade), Resazurin sodium salt (AlamaBlue, Bioreagent), ammonia hydroxide solution (25%), L-phenylalanine (Phe, 98.5-101.0%), L-leucine (Leu, 98.5-101%), L-tryptophan (Try, 99-101%), L-histidine (His, 99%), L-threonine (Thr, 98%), L-methionine (Met, 98%), L-isoleucine (Ile, 98%), L-lysine (Lys, 98%), L-valine (Val, 98%), N-hydroxysuccinimide (NHS, 98%), Fluorescein isothiocyanate isomer I (FITC), 2-amino-2-norbornanecarboxylic acid (BCH), cis-Diammineplatinum(II) dichloride (Cisplatin, European Pharmacopoeia (EP) Reference Standard) were all purchased from Sigma-Aldrich. N-(3-Dimethylaminopropyl)-N′-ethylcarbodiimide hydrochloride (EDC chloride, purum, >98%) was purchased from Fluka. CellROX® Orange, Hoechst 33342, goat anti-Mouse IgG secondary antibody conjugated with Alexa Fluor 488 were purchased from Invitrogen. LAT-1 (D-10) mouse monoclonal IgG1 primary antibody was purchased from Santa Cruz Biotechnology. Caspase 3, Caspase 8 and Caspase 9 Multiplex Activity Assay Kit (Fluorometric) was purchased from Abcam. Z-IETD-FMK (ALX-260-144-R100, Caspase 8 inhibitor) and Z-VAD-FMK (ALX-260-138-R100, Caspase 3 inhibitor) were purchase from Enzo Life Sciences. Dulbecco's Modified Eagle's Medium (DMEM-High glucose), Roswell Park Memorial Institute (RPMI)—1640 Medium dry powder, Fetal Bovine Serum (heat inactivated), and Antibiotic-Antimycotic were purchased from GE Hyclone. Trypsin (0.25%, with 1 mM EDTA, 4 Na) was purchased from Gibco. All chemicals were used without further purification. Phosphate-buffered saline (PBS) was used to prepared nanoparticles dispersion for surface functionalization. Deionized (DI) water was used to prepared nanoparticle stock suspension for in-vitro and vivo study.

### Synthesis of Nano-PAAM

To synthesize amino-functionalized mesoporous Si nanoparticles (NH₂-MSN) with a primary size of 30 nm, CTAB (4 mM) was dissolved in DI water (140 ml) with vigorous stirring at 40° C. Next, APTES and TEOS were mixed with a molar ratio of 1:4 to make up a total amount of 2 g, which was followed by dropwise addition into CTAB aqueous solution. After 15 min for stabilizing the reactants, NH₃.H₂O (25%, 0.5 ml) was added and the reaction was continued for another 5 h at 40° C. with vigorous stirring. To synthesize NH₂-MSN with a size of 80 nm, 170 nm or 260 nm, the amount of NH₃.H₂O remained unchanged in the case of NH₂-MSN (80 nm) but was increased to 3 or 5 ml respectively in the case of NH₂-MSN (170 nm) and NH₂-MSN (260 nm). Additionally, the synthesis temperature for the above-mentioned three NH₂-MSN variants was reduced to room temperature while other factors remained unchanged. Upon completion of the reaction, the product was extracted via centrifugation at 8000 rpm and washed with 95% ethanol for 3 times. To obtain mesoporous structure, the as-synthesized Si nanoparticles were dispersed in 140 ml of solution containing ethanol (95%) and 1M HCl (v/v=1). The dispersion was then kept at 60° C. for 24 h with vigorous stirring. The final product was subject to two-step centrifugation (1^{st} 2000 rpm, 5 min; 2^{nd} 12000 rpm, 5 min) in order to remove any bulky aggregates formed during the reaction. The particles were air-dried overnight at 50° C. and stored in a desiccator at room temperature for further use.

The non-porous version of Si nanoparticles (SiNPs) with a size of 30 nm was synthesized as follows: mixture of solution containing 3 ml NH₃.H₂O, 5.4 ml TEOS, 2.6 ml H₂O, and 100 ml absolute ethanol was prepared and vigorously stirred at room temperature for 5 h. After that, the unmodified SiNPs were collected by the sequential steps including ample washing, centrifugation, air dry as described above. Next, amino functionalization was realized by adding 48 μl APTES to the SiNPs solution (in 50 ml Abs ethanol) with a molar ratio of 1:4. The reaction was continued at 75° C. for 6 h. The NH₂-SiNPs were collected following the previously described protocol.

Nano-PAAMs were synthesized via the well-established EDC-NHS coupling reaction. In brief, 20 μmol each type of EAAs, 30 μmol EDC, and 50 μmol NHS were mixed and dissolved into 10 ml PBS buffer. The mixture was later stirred vigorously for 30 min for a complete activation of EDC prior to addition of the nanoparticles. 10 mg of the as-synthesized NH₂-MSNs or NH₂-SiNPs were suspended in 20 ml PBS and sonicated in an ice-covered ultrasonic bath for at least 30 min to ensure a well-dispersed nanoparticles suspension. Next, the suspension was added into the above-made coupling solution and the reaction was kept at room temperature for 24 h with vigorous stirring. The final product was extracted via centrifugation at 12000 rpm and amply washed by distilled water. Finally, the particles were freeze-dried overnight and stored in the desiccator for further use.

### Transmission Electron Microscopy

The primary size of solid/ mesoporous silica nanoparticles with different conjugated amino acids was characterized using transmission electron microscope (TEM, Carl Zeiss Libra 120 Plus). To prepare the sample for TEM imaging, 30 μl of nanoparticles solution (100 μg/ml) suspended in absolute ethanol was pipetted onto a carbon-coated copper grid and the samples were air-dried at room temperature overnight. The copper grid containing samples were then placed into the sample holder and inserted into TEM for imaging. The voltage was set to be 120 kV and magnification was adjusted in a range from 10000 to 70000 in order to obtain images of fine quality.

### Dynamic Light Scattering and Zeta Potential Analysis

To measure the size distribution and surface charge of various nanoparticles, hydrodynamic diameter (D_{H}) and zeta potential (ζ) of each type of nanoparticles were characterized by the Zetasizer Nano ZS (Malvern). In order to obtain a desirable suspension of the nanoparticles, the nanoparticles (1 mg/ml) were sonicated at least for 30 min in ice bath. To better characterize the physiochemical properties of the nanoparticles in the biological milieu, D_{H} and ζ of the nanoparticles in the complete cell culture medium dispersant were also analyzed. In brief, the nanoparticles (1 mg/ml) were suspended in cell culture medium with 30 min of sonication in ice bath. The suspension was then placed into incubator for 30 min followed by centrifugation at 12000 rpm to retrieve the nanoparticles. The nanoparticles were then resuspended into DI water following the above preparation step and examined by Zetasizer Nano Zs (Malvern) for D_{H} and ζ.

### Fourier-Transform Infrared Spectroscopy (FTIR)

FTIR was employed to confirm the successful surface modification of various nanoparticles. Briefly, approximately 0.5 mg nanoparticles with various functional groups were weighed and thoroughly blended with potassium bromide in an agate mortar via vigorous grinding. The mixture was then decanted into a mould for compression to form an ultrathin film. The film was then loaded into a FTIR machine for examination. Results were subject to calibration by the machine to remove background noise.

### Cell Culture and Cytotoxicity Assay

MDA-MB-231, MCF-7, II-4, HDF, NCM-460, and HaCaT were cultured in DMEM supplemented by 10% FBS and 1% antibiotics. MKN was cultured in RPMI1640 DMEM supplemented by 10% FBS and 1% antibiotics. The cells were routinely maintained in a cell culture incubator (Thermo) at 37° C., 5% CO₂ and 95% relative humidity. Cell morphology and confluency were visualized using Carl Zeiss Primo Vert inverted bright field microscope. Upon confluence, cells were trypsinized and seeded into 96 well plates with an optimal seeding density to obtain 70% confluence prior to cell viability measurements. Thereafter, the cells were treated with either various nanoparticles or cisplatin at various concentrations for further 24 h. MDA-MB-231 cancer spheroids were formed as described in our earlier paper (Su, H. et al., Acta Pharm Sin B 2019, 9 (1): 49-58). Cell viability was determined using the alamarBlue® cell viability assay. After 2 h of incubation with alamarBlue® in cell culture incubator, raw data was obtained from the high-throughput microplate reader (Molecular Devices SpectraMax M2) with maxima wavelength set i.e. ex/em 530/590 nm.

### Annexin V/PI Apoptosis Assay

Cells were trypsinized and washed with annexin V binding buffer 2 times before the cells were further incubated in a binding buffer consisting of 5 μl of FITC-Annexin V and 2 μl of PI (100 μg/ml) for 15 mins at 4° C. in the dark. Thereafter, the cells were washed extensively with PBS to remove any excess dyes, the stained samples were then subjected to both fluorescence imaging as well as flow cytometry analysis. For microscope imaging, the protocol has been described above. For flow cytometry, please refer to Flow cytometry for more detailed information.

### Reactive Oxygen Species Measurement

CellROX® Orange reagent was used to detect the intracellular ROS level in MDA-MB-231 cells. Upon confluence, cells were exposed to 500 μg/ml of NH₂-MSN, or Nano-pPAAM or Nano-SiNP for 8 h, followed by addition of 0.2 μl of CellROX® Orange reagent and 10 μl of Hoechst 33342 (10 μg/ml) in cell culture medium. MDA-MB-231 cells treated with tert-Butyl hydroperoxide (TBHP) (100 μM) for 2 h serves as a positive control. All the samples were then incubated at 37° C. for 0.5 h, washed with serum-free culture media to remove the excess dyes, and finally imaged via fluorescence microscope (Carl Zeiss AxioObserver Z1). Intracellular ROS expression level was quantified with the ImageJ software.

### Immunostaining

Samples were fixed with 4% paraformaldehyde for 15 min at room temperature and the cells were further permeabilized using TritonX (0.2%) for 10 min. Thereafter, the samples were washed 3 times with PBS, and further blocked with 2% BSA (blocking buffer) for 1 h at room temperature. LAT-1 mouse monoclonal primary antibody was diluted in the blocking buffer (1:100) and incubated with the cell samples overnight at 4° C. Following which, the samples were washed 3 times with PBS, counterstained with 10 μg/ml Hoechst 33342, 66 nM rhodamine phalloidin and goat anti-mouse IgG secondary antibody conjugated with Alexa Fluor 488 (1:200 v/v) for 1 h at room temperature. Samples were then imaged using fluorescence microscope (Carl Zeiss AxioObserver Z1).

### Cellular Uptake Studies

To investigate whether Nano-pPAAM was internalized by MDA-MB-231 cells via endocytosis, FITC was further conjugated to the Nano-pPAAM following the established protocol described elsewhere with slight modification (Guo, H. et al., Chem Cent J 2011, 5 (1): 1). In brief, 50 mg dry nanoparticles were added into 150 ml PBS buffer followed by addition of 2 ml FITC solution (1 mg/ml in DMSO). The mixture was then vigorously stirred in the dark for 6 h at room temperature. The final product was air-dried overnight at 50° C. preceded by ample wash with absolute ethanol and centrifugation at 12000 rpm for 10 min.

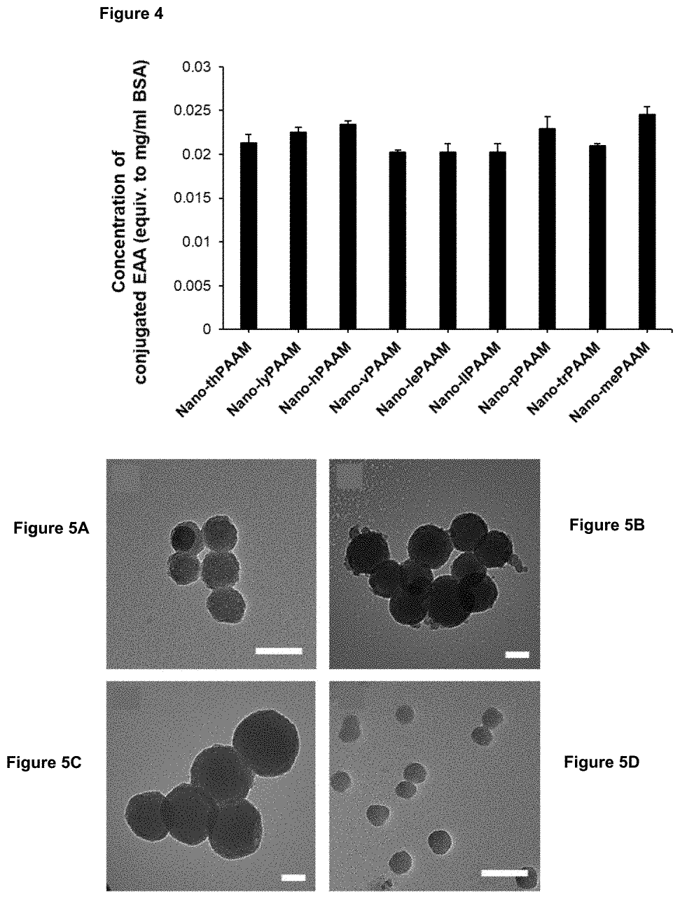

Upon confluence, cells were exposed to 500 μg/ml of the FITC-conjugated Nano-pPAAM for 4 h under various conditions (refer to FIG. 4). Following which, the unbounded excess NPs was removed by washing the samples with serum-free culture media before the cells were trypsinized and collected. Next, cell nuclei were stained with 10 μg/ml Hoechst 33342 for 10 min at 4° C. FITC signals resulting from the particles that reside on the external side of plasma membrane were quenched using 0.2 mg/ml trypan blue solution to minimize background signal interference. Finally, 50 μl of each sample was then mounted onto glass slides and imaged with the fluorescence microscope (Carl Zeiss AxioObserver Z1).

### Inductively Coupled Plasma Mass Spectrometry (ICP-MS)

To investigate whether Nano-pPAAM utilizes LAT-1 as its unique endocytosis pathway, MDA-MB-231 cells upon confluence were cotreated with 10 mM BCH and Nano-pPAAM (200 μg/ml) or NH₂-MSN (200 μg/ml) for 6 h before they were harvested by 4M NaOH. Cell counting using hemocytometer was conducted prior to the harvest by NaOH for normalization purpose. Full digestion of the silica nanoparticles into Si ion was ensured by vigorously stirring the sample solutions at 300 rpm for 24 h at room temperature. A proper dilution by diluted HCl (1M) was required to prepare the testing solution (PH˜7) for the ICP-MS analysis. Uptake of Nano-pPAAM or NH₂-MSN was calculated by the concentration of the Si in the testing solution which would then be normalized by the cell number in each sample and the data was presented as Si concentration (ppb)/cell.

### Quantitative Real-Time Polymerase Chain Reaction (RT-PCR)

Total RNA was isolated using PureLink RNA Mini Kit (Life Technologies). Reverse transcription of RNA samples was done with the iScript cDNA synthesis Kit from Biorad, in accordance to the manufacturer's protocol. RT-PCR was conducted using the CFX96 real time PCR detection system from Biorad and SYBR FAST qPCR Master Mix (2×) Universal from KAPA with the following thermal cycling condition: Enzyme activation at 95° C. for 3 min; followed by 40 cycles of denaturation at 95° C. for 3s and annealing/extension/data acquisition at 60° C. for 20 s. Melt-curve analysis was also done to assess the purity of the amplicon/ product. All the primers were verified via primer bank (https://pga.mgh.harvard.edu/primerbank/) prior to purchase from Sigma Aldrich and listed in Table 1.

TABLE 1
List of primers for PCR.

| | RT-PCR primer sequence | |
|---|---|---|
| Gene Target | Sense | Antisense |

Housekeeping gene

| | | |
|---|---|---|
| GAPDH | GGAGCGAGATCCCTCCAAAAT | GGCTGTTGTCATACTTCTCATGG |
| | (SEQ ID NO: 1) | (SEQ ID NO: 2) |

Apoptotic genes

| | | |
|---|---|---|
| Caspase 3 | TACCTCTTATGAGGAGAAACGGT | AGGAAAGTCCAGGTCTAGCTTG |
| | (SEQ ID NO: 3) | (SEQ ID NO: 4) |
| Caspase 6 | CATGCTGGGAAGATACTGTTGAT | GCCCGAGACTAACAAAAGACTCT |
| | (SEQ ID NO: 5) | (SEQ ID NO: 6) |
| Caspase 8 | GGGAGCCTCTTGCAGGATAAA | GAATGGGGCATAGCTCACCAC |
| | (SEQ ID NO: 7) | (SEQ ID NO: 8) |
| Caspase 9 | TGTCTTGGAATGCACTGTATCTC | CCCAGTAAGGCTGTAAATGCTC |
| | (SEQ ID NO: 9) | (SEQ ID NO: 10) |
| Caspase 12 | TGGAGCTGGTAACCCAGTAGG | TGGTACCTTTGCCTTGGAGTATT |
| | (SEQ ID NO: 11) | (SEQ ID NO: 12) |

### Flow Cytometry

Cells post Nano-pPAAM treatment were trypsinized and washed with PBS 3 times prior to addition of caspase 8 staining buffer. 100 μl of each sample was transferred to 96 well plate and loaded in the Guava InCyte™ 3.0. The machine was subject to proper cleaning before examination of the samples. A blank sample (unstained cells) was used for calibration and gating purpose. The flow rate was set to 12 μl per min. Experiments were conducted in triplicate. This protocol also applies to annexin V/PI apoptosis assay.

Anticancer effectiveness of Nano-pPAAM in xenografted mice model,

Six-week-old male NSG mice (Jackson Laboratories, Sacramento, Calif., USA) were subcutaneously injected with 2×10⁵ MDA-MD-231 cells. Intratumoral injection of MSN (100 μL of 3 mg/mL) started 1 week after tumor xenograft, when the xenograft was palpable. The xenograft was harvested and weighted two weeks after treatment. Power analysis was used to determine sample size, Double-blind randomization was used for allocation of the experimental groups. All animal experiments were carried out in accordance to the guidelines of the Institutional Animal Care and Use Committee (ARF-SBS/NIE-A0250AZ, -A0324 and -A0321) of Nanyang Technological University, Singapore.

For histological analysis, the harvested tumors and the target organs (i.e. heart, liver, kidney, and lung) were fixed in 4% PFA overnight. Next, the fixed tissues were washed with PBS and dehydrated with series of ethanol—70%, 80%, 90%, two changes of 100%, 1 h each and overnight at 100%. The next day, they were followed by two changes of xylene, for 3 h and overnight. Then they were infiltrated with paraffin wax for overnight before embedding into cassette blocks. 5 μm thin sections of the tissues were made by microtome and the sections were attached onto the glass slides. The slides were kept at 37° C. for proper attachment of tissue section. Thereafter, the glass slides were dewaxed with two changes of xylene and rehydrated with descending series of ethanol (100% to 70%), 5 minutes each. The slides were rinsed in tap water for 5 mins and stained with hematoxylin dye for 5 to 10 minutes, followed by 30 seconds wash in running tap water. The slides were then dipped in acid alcohol (70% ethanol, 1% Hydrochloric acid (37%), 29% DI water) for 15 seconds and again washed for 30 seconds. They were then placed in Scott's tap water (2 g Sodium Bicarbonate, 20 g Magnesium sulfate in 1 L DI water) for 5 minutes followed by 30 seconds wash. Finally, they were stained with Eosin dye for 5 to 10 minutes and again washed. After staining, the slides were dehydrated following the ascending series of alcohol (70% to 100%). They were mounted with Cytoseal or DPX mounting medium and allowed to dry overnight before microscopic analysis.

To examine the particle distribution in different organs/tumor of the tumor-bearing scid mice, the dissected tumors and organs subjected to different treatments were digested using concentrated NaOH (4M) instead of fixation by 4% PFA at 60° C. overnight. Upon completion of the digestion process, testing samples solution was prepared as described earlier in the Inductively coupled plasma mass spectrometry (ICP-MS) section and the particle concentration in various samples were quantified via ICP-MS.

### TUNEL Assay

To examine whether the Nano-pPAAM treated tumor in the mice was undergoing apoptosis, we used APO-BrdU™ TUNEL Assay Kit (Invitrogen™) to immunostain the tumor microtome sections as prepared previously. A slight modification of the company-provided manual was applied to yield the optimal results. Briefly, tumor sections were rinsed by wash buffer to enhance its wettability in order for a better spread-out of the DNA-labeling solution. 50 μl of DNA-labeling solution (consisting of 10 μl of reaction buffer, 0.75 μl of TdT enzyme, 8 μl of BrdUTP, and 31.25 μl of DI H2O) was prepared for each sample. Next, the as-prepared DNA labeling solution was added onto the sections on top of which a coverslip was applied. The DNA-labelled tumor sections were then placed in the dark at room temperature overnight. Upon completion of the DNA labelling, samples underwent ample washing with rinse buffer. Following which was the addition of antibody staining solution (including 5 μl of the Alexa Fluor™ 488 dye-labeled anti-BrdU antibody, 5 μl of propidium iodide/RNase A staining buffer, and 90 μl of rinse buffer) onto each sample. The staining lasted for 1 h in the dark at room temperature. The antibody-bound samples were then imaged by fluorescence microscope (Carl Zeiss AxioObserver Z1).

### Statistical Analysis

All experiments in this study were carried out with triplicates. Data are presented by mean±standard deviation (SD) with p value indicated where necessary. Origin 9 (OriginLab) was used for statistical analysis. Experimental data were subjected to either Student's t-test or one-way analysis of variance (ANOVA) where applicable. Statistical differences are indicated with probability value (p value) in the associated text or figure legend.

### Example 2

### Characterization and In Vitro Screening of Panel EAA Conjugated Mesoporous Silica Nanoparticles

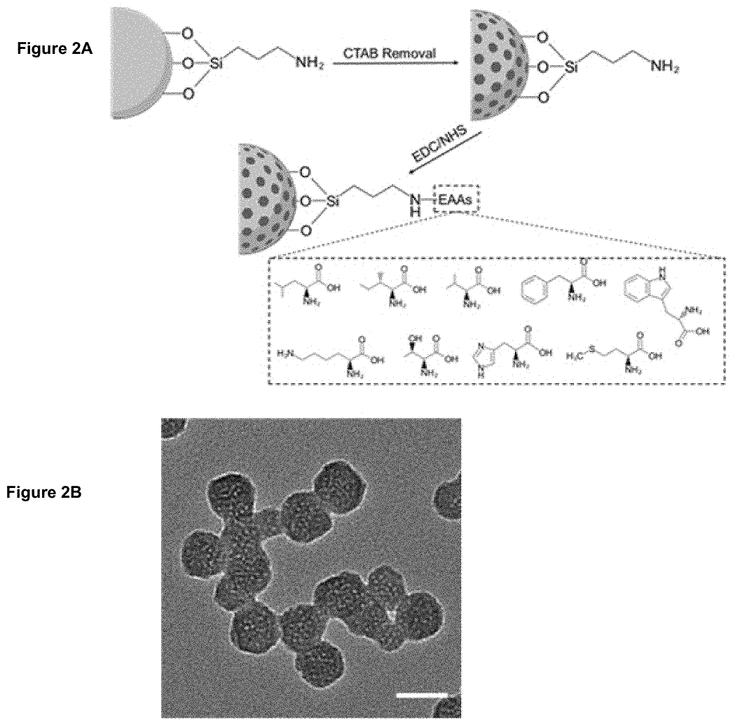

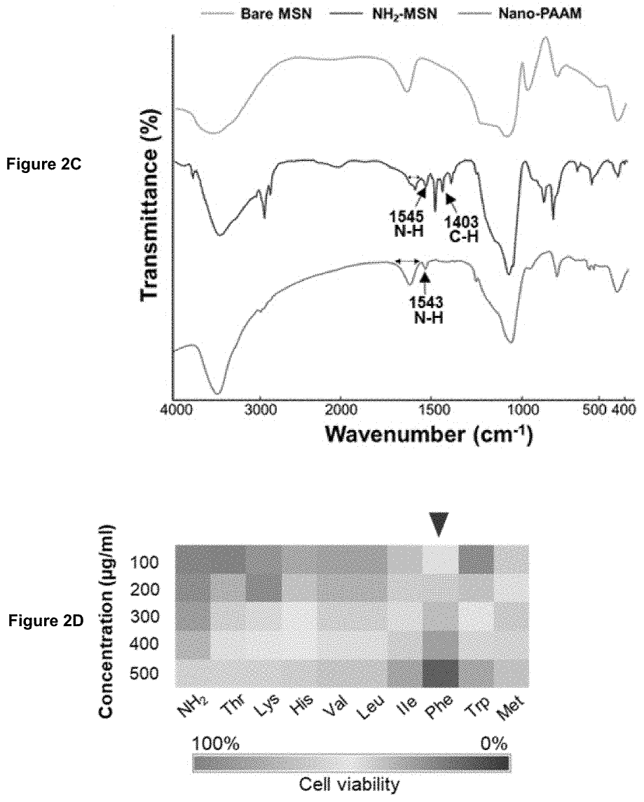

Amino-functionalized mesoporous nanoparticles (NH₂-MSN) were synthesized via a modified classical Stóber method followed by removal of the cetyl trimethyl ammonium bromide (CTAB) template at ˜pH 1 (FIG. 2A)(Hao, N. et al., ACS Appl Mater Interfaces 2015, 7 (2): 1040-5). Thereafter, the panel EAA (i.e. leucine (Leu), tryptophan (Trp), lysine (Lys), methionine (Met), threonine (Thr), valine (Val), histidine (His), isoleucine (Ile) and L-phenylalanine (Phe)) were conjugated onto the surface of the MSN core at equivalent concentration via the ethyl(dimethylaminopropyl) carbodiimide (EDC)N-Hydroxy succinimide (NHS) coupling reaction to produce a small library of Nano-PAAMs (Bartczak, D. and Kanaras, A. G., Langmuir 2011, 27 (16): 10119-23). The TEM image of Phe-conjugated nano-PAAM (Nano-pPAAM), which bears the size and shape representative of the other Nano-PAAMs prepared in this study is shown in FIG. 2B. The Nano-PAAMs employed in this study were revealed to possess wormhole-like mesopore (˜2-3 nm) structure and a primary particle size of approximately 30 nm. Successful surface functionalization of the porous nanoparticles was determined with Fourier-transform infrared spectroscopy (FTIR). Peaks at 1545 cm⁻¹ as well as 1403 cm⁻¹ were detected in the amine-functionalized (NH₂-MSN) sample, which could be attributed to the N—H bending and C—H stretching vibration respectively (FIG. 2C) (Peña-Alonso, R. et al., Journal of Materials Science 2006, 42 (2): 595-603). Upon conjugation of EAA to the MSN surface, a broader peak at approximately 1630 cm⁻¹ could be detected, which corresponds to the coexistence of Si-O and C═O upon the formation of the amide group (Ahmed A. and Moosa, B. F. S., American Journal of Materials Science 2017, 7 (6): 223-231). EAA conjugation efficiency on the MSN was determined spectroscopically at 205 nm, which corresponds to the absorbance of the amide bond (Ahmed A. and Moosa, B. F. S., American Journal of Materials Science 2017, 7 (6): 223-231). As depicted in FIG. 4, which shows the BSA-equivalent concentrations of the surface bound EAA on the MSN, we did not observe any significant difference in terms of EAA conjugation efficiency amongst the different EAA-MSN variants. Next, dynamic light scattering (DLS) measurement revealed that the hydrodynamic diameters (D_{H}) of the Nano-PAAM variants are less than 200 nm in either DI water or cell culture medium (Table 2).

TABLE 2
Size and surface charge measurement of the various
Nano-PAAMs in different dispersants.

| | | Hydrodynamic diameter | | Zeta potential | |
|---|---|---|---|---|---|
| | Primary size | [nm]±SD | | [mv]SD | |
| Sample | [nm]±SD | H₂O | DMEM | H₂O | DMEM |
| NH₂-MSN | 32.6±5.5 | 78.3±7.4 | 94.9±24.8 | 39.2±6.9 | −20.1±0.1 |
| Nano-lePAAM | 39.7±7.8 | 85.5±4.2 | 154.9±25.2 | 24.4±7.2 | −18.2±5.3 |
| Nano-trPAAM | 38.9±8.8 | 90.5±16.8 | 117.9±34.9 | 29.6±4.1 | −9.6±4.0 |
| Nano-lyPAAM | 38.7±11.5 | 90.1±49.4 | 112.4±6.1 | 27.5±1.5 | −13.9±0.3 |
| Nano-mePAAM | 29.2±5.5 | 174.8±37.9 | 124.8±21.6 | 20.1±0.6 | −18.5±0.2 |
| Nano-thPAAM | 33.9±6.4 | 134.8±27.7 | 118.4±46.9 | 29.3±1.4 | −17.8±0.5 |
| Nano-vPAAM | 29.2±5.5 | 108.9±53.8 | 109.0±42.1 | 26.6±0.7 | −18.7±1.1 |
| Nano-hPAAM | 27.4±4.9 | 99.1±34.4 | 119.3±14.1 | 18.8±0.6 | −17.4±0.5 |
| Nano-IIPAAM | 37.3±9.0 | 79.8±31.4 | 118.1±4.5 | 18.6±0.5 | −17.0±1.2 |
| Nano-pPAAM | 30.0±5.7 | 127.5±15.2 | 152.5±17.7 | 32.5±11.6 | −18.2±6.4 |

Zeta potential measurements revealed a positively charged surface (+20-40 mV) for the NH₂-MSN and Nano-PAAM groups in DI water, which could be accounted for by the presence of amino moieties on the MSN surface. Conversely, we observed a surface charge reversal of the particles in the cell culture medium, which is indicative of the formation of the protein corona covering the particles (Wu, Z. et al., Nanotoxicology 2018, 12 (10): 1215-1229).

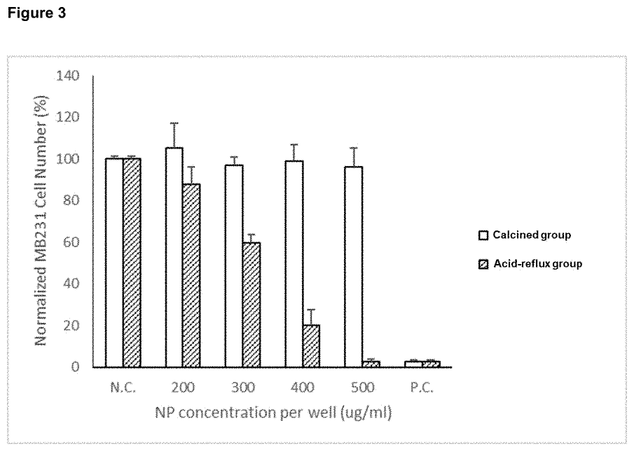

We next conducted a non-bias in vitro screening of the Nano-PAAM panel using the MDA-MB-231 human breast cancer cell line. As shown in FIG. 2D, it was noted that the NH₂-MSN treatment only has a marginal effect on cell viability (<20%) even at the highest concentration of 500 μg/ml. This observation is consistent with numerous other studies showing that MSN is a non-toxic and cyto-compatible material (Pandele, A. M. et al., Materials (Basel) 2018, 12: (1); Yang, Y.-W., MedChemComm 2011, 2: (11)). In stark contrast, a significant dose-dependent decrease in the cancer cell viability by approximately 2 to 6-fold was observed in the Nano-PAAMs treated groups compared to the NH₂-MSN group (FIG. 2D). Interestingly, the anti-cancer effects of the Nano-PAAM variants were observed to scale according to the hydrophobicity of the conjugated EAA side chain. Specifically, compared to the polar EAAs (i.e., Thr, Lys, His), Nano-PAAM functionalized with hydrophobic EAA such as Trp, Ile, Met and Phe (Zhu, C. et al., Proc Natl Acad Sci U S A 2016, 113 (46): 12946-12951), were observed to exhibit enhanced cancer-killing efficacy. This observation corroborates with earlier studies, demonstrating that peptides with higher hydrophobicity could perturb the long-range ordering of the membrane in cancer cells by promoting the hydrophobic-hydrophobic interaction between the peptides and the lipid bilayer (Deber, C. M. and Stone, T. A., Peptide Science 2019, 111: (1); Huang, Y. B. et al., Mol Cancer Ther 2011, 10(3): 416-26; Tauqir, N. A., Journal of Environmental & Agricultural Sciences 2016, 9: 14). Furthermore, while it was previously shown that melanoma targeting peptide conjugated drug-free silica nanoparticles (˜6-10 nm) could kill cancer cells under amino acids/serum deplete culture condition via the process of ferroptosis, Nano-PAAMs remain efficacious even in the presence of serum (Kim, S. E. et al., Nat Nanotechnol 2016, 11(11): 977-985). Noteworthy, among the Nano-PAAMs screened, Nano-pPAAM was observed to elicit the most potent anti-cancer effect, with a maximal killing rate of ˜80% at the highest concentration (500 μg/ml) probed (FIG. 2D arrowhead). Interestingly, we further uncovered that only the Nano-pPAAM prepared using the acid reflux method, but not the calcination method, could possess the anti-cancer property (FIG. 3). Therefore, through our screening effort, Nano-pPAAM was revealed as a novel prototypical Nano-PAAM nanotherapeutics that is endowed with potent intrinsic anti-cancer properties.

### Example 3

### Cellular Uptake and LAT-1 Receptor Targeting Properties of Nano-pPAAM

Since the physicochemical properties of NPs are known to be key determinants of NPs induced biological outcomes (Luo, Z. et al., Macromol Rapid Commun 2019, 40(5): e1800029; Reisch, A. et al., Advanced Functional Materials 2018, 28(48)], we investigated whether changing the particle size of Nano-pPAAM would potentially alter its anti-cancer efficacy. To determine the effects of particle size, Nano-pPAAM with different core particle sizes (i.e. 80, 170, and 260 nm) were synthesized and their anti-cancer activities were compared to the 30 nm variant. The representative TEM images, as well as the D_{H} and zeta potential of the bigger sized Nano-pPAAM are shown in Table 3 and FIG. 5, respectively.

TABLE 3
Size and surface charge measurement of the various silica NPs in different
dispersants.

| | Primary size | | Hydrodynamic | | Zeta potential | |
|---|---|---|---|---|---|---|
| | [nm] ± SD | | diameter [nm] ± SD | | [mv] ± SD | |
| Sample | Theoretical | Measured | H₂O | DMEM | H₂O | DMEM |
| NH₂- | 80 | 76.1 ± 11.8 | 129 ± 29 | 139 ± 43.3 | 38.2 ± 4.5 | −21.8 ± 0.6 |
| MSN | 170 | 170.3 ± 41.8 | 139.5 ± 9.8 | 171.1 ± 46.1 | 38.6 ± 0.4 | −18.2 ± 0.6 |
| | 260 | 258.5 ± 33.7 | 250.5 ± 24.1 | 286.9 ± 53.6 | 35.1 ± 0.4 | −18.4 ± 0.6 |
| Nano- | 80 | 82.6 ± 12.7 | 106.6 ± 21 | 215.1 ± 47.5 | 21.1 ± 0.5 | −18.3 ± 1.5 |
| pPAAM | 170 | 165.9 ± 19.9 | 181.9 ± 4.9 | 134.7 ± 24.8 | 32.3 ± 1.4 | −20.1 ± 1.1 |
| | 260 | 256 ± 21.6 | 288.2 ± 8.6 | 273.4 ± 34.9 | 44.1 ± 1.2 | −21.1 ± 0.7 |
| Nano- | 30 | 37.8 ± 5.4 | 169.2 ± 13.6 | 182.1 ± 23.8 | 1.95 ± 9.7 | −27.6 ± 4.4 |
| pSiNP | | | | | | |

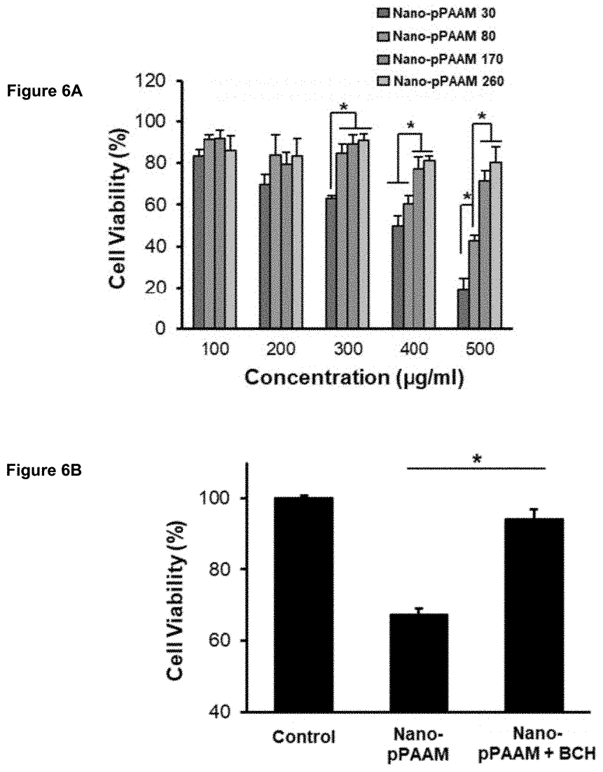

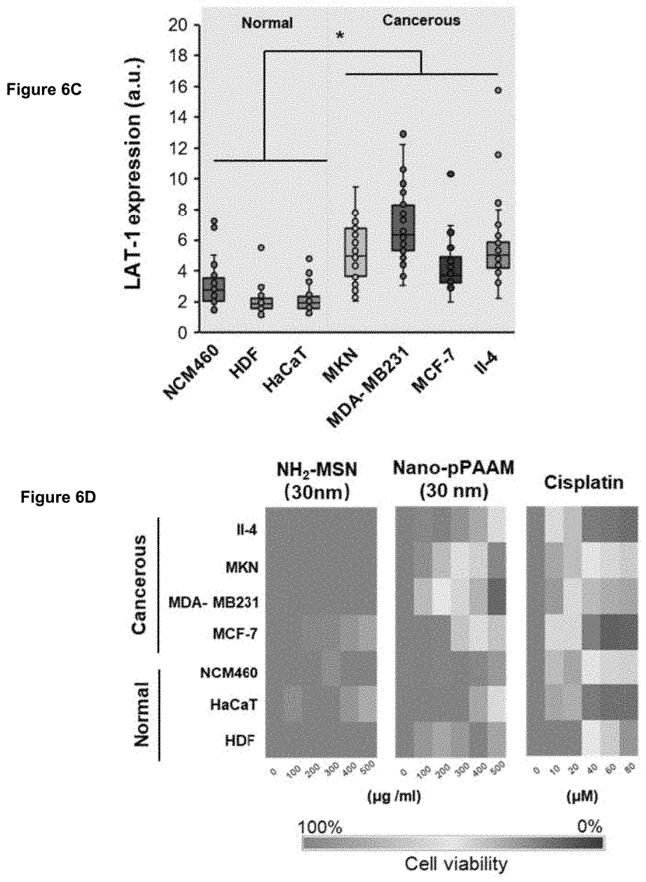

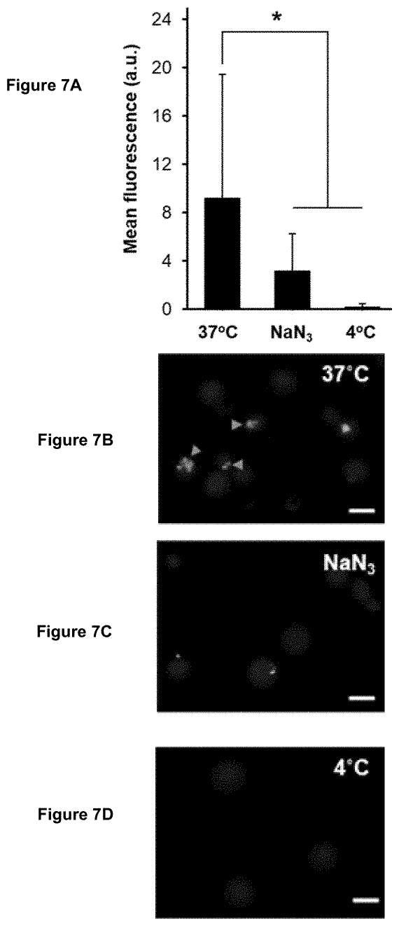

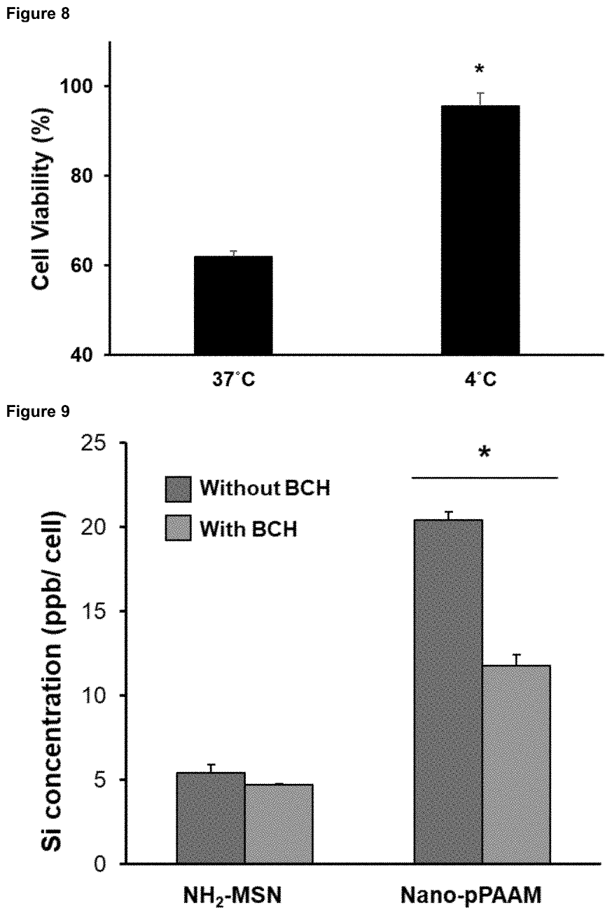

Increasing the primary particle size to 80 nm (i.e. Nano-pPAAM 80) resulted in a slight (23%) but significant decrease in anticancer efficiency compared with Nano-pPAAM 30 (FIG. 6A). However, when the particle size was further increased to 170 nm and 260 nm, the anti-cancer capability was effectively muted, suggesting that an optimal range of particle size was necessary to induce a potent anti-cancer effect. A possible explanation could be that Nano-pPAAM is internalized by endocytosis and that the process is favored at certain size range (10-60 nm), which in our study was found to be 30 nm (Zhang, S. et al., Adv Mater 2009, 21: 419-424; Huang, C. et al., Nano Lett 2013, 13(9): 4546-50]. Indeed, pre-treating the cells with sodium azide (10 mM), a potent endocytosis inhibitor, or exposing the cells to Nano-pPAAM at 4° C., resulted in a significant reduction of particle uptake into the cell cytoplasm (FIG. 7). Importantly, we further showed that inhibiting the endocytosis of Nano-pPAAM by lowering the treatment temperature to 4° C., can substantially decrease the NP-induced cell death by as much as 40% within 6 h of treatment time as opposed to the experiment conducted at 37° C. (FIG. 8). Collectively, these findings strongly suggest that Nano-pPAAM entry into the cells is an energy-dependent process and necessary to elicit the NPs induced cancer killing effect.

To probe deeper into the uptake mechanism of Nano-pPAAM by the MDA-MB-231 cells, we next turned our focus to another endocytic machinery specific for amino acid uptake. The system L is a major nutrients transport system that is responsible for the conveyance of large neutral amino acids and several essential amino acids (EAA) into the cells (Fotiadis, D. et al., Mol Aspects Med 2013, 34(2-3): 139-58). Among the 4 sub-types of L amino acid transporters (LAT 1-4), LAT-1, a sodium-independent exchanger for amino acids, exhibits specific functional features that are associated with cancer cells. LAT-1 forms a heterodimeric complex with 4F2 cell surface antigen (CD98) to facilitate the transport of neutral EAAs such as Val, Leu, Ile, Phe (Geier, E. G. et al., Proc Natl Acad Sci U S A 2013, 110(14): 5480-5). Just like other surface-bound transporters, LAT-1 can be trafficked and recycled via the endocytic machinery, thereby regulating its expression on the cell surface (Li, L. et al., Nanomedicine 2017, 13(3): 987-998). Furthermore, biogenesis of LAT-1 may occur at the endoplasmic reticulum (ER) to ensure that supply of LAT-1 can keep pace with its cellular demand (Saftig, P. and Klumperman, J., Nat Rev Mol Cell Biol 2009, 10(9): 623-35; Scalise, M. et al., Biochim Biophys Acta 2016, 1857(8): 1147-1157). Specifically, LAT-1 plays a key role to supply EAA to growing tumour cells by activating pro-growth signalling pathways such as the mammalian target of rapamycin (mTOR) (Hayase, S. et al., Oncol Lett 2017, 14(6): 7410-7416). To examine whether uptake of Nano-pPAAM could be mediated via LAT-1, we attempted to block its intracellular entry by using the LAT-1 inhibitor, 2-Amino-2-norbornanecarboxylic acid (BCH). Inductively coupled plasma mass spectrometry (ICP-MS) was employed to determine the intracellular content of Si in MDA-MB-231 cells with and without BCH (10 mM). The amount of internalized Si detected in the Nano-pPAAM group was 20.4 ppb/cell, while the intracellular Si level in the BCH (10 mM) treated cells was significantly reduced by approximately 2-fold (Si: 11.7 ppb/cell) (FIG. 9). In contrast, there was no detectable changes in the uptake of NH₂-MSN in the BCH-treated cells as compared to that without BCH treatment. This observation, coupled with the favourable internalisation (˜4-fold) of Nano-pPAAM over its amino functionalized counterpart, further lends support to our hypothesis that Nano-pPAAM targets the LAT-1 receptor. Our findings corroborated previous studies, showing that sub-200 nm nanoparticles can interact and enter the cells via LAT-1 (Su, H. et al., Acta Pharm Sin B 2019, 9(1): 49-58). The importance of LAT-1 for Nano-pPAAM to exert its anticancer property was also demonstrated by a marked decrease in the cancer-killing efficacy of Nano-pPAAM when its entry to the cells through LAT-1 was blocked. Specifically, the inhibition of LAT-1 by BCH completely abolished the apoptosis-inducing action of Nano-pPAAM on MDA-MB-231, reaching a level that was comparable to the untreated control group (FIG. 6B).

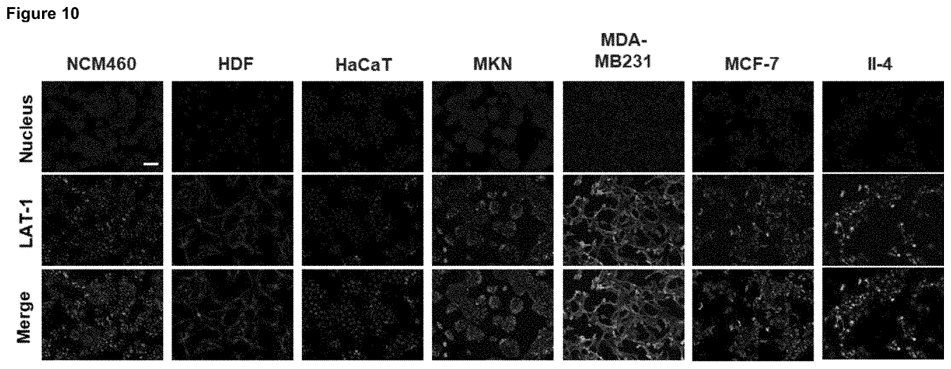

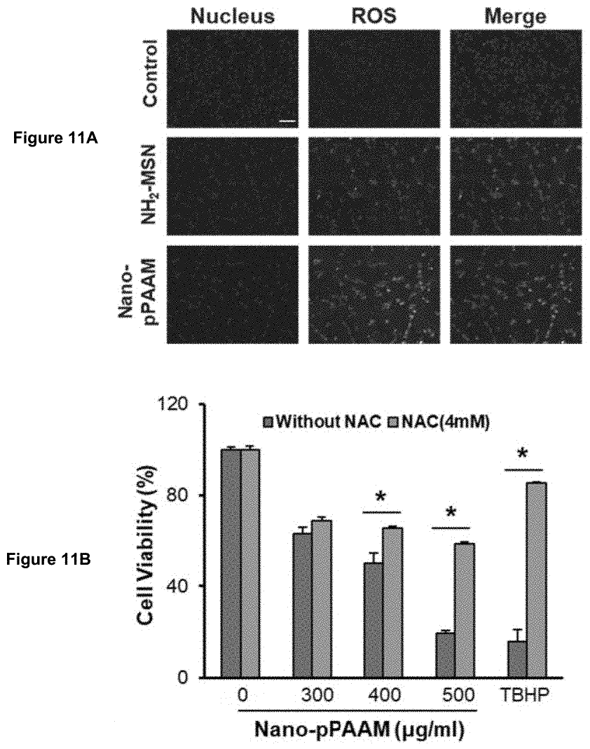

Since overexpression of LAT-1 is a hallmark of several cancers (Hafliger, P. and Charles, R. P., Int J Mol Sci 2019, 20(10): 2428), we hypothesized that the uptake of Nano-pPAAM via LAT-1 could be exploited to achieve selective targeting of cancer cells. To examine this possibility, we extended our in vitro screening regime to an additional panel of cancer cells such as MCF-7 (breast cancer), MKN (gastric cancer), II-4 (HaCaT ras clone), as well as non-cancerous cells such as HaCaT (skin), HDF (skin), NCM-460 (colon). The presence of LAT-1 in these cell lines was first assessed by immunocytochemical staining. As expected, enhanced expression of LAT-1 in the cancer cells (˜2.5-fold) was evident when compared to the non-cancerous cell lines, (FIGS. 6C and 10). While the NH₂-MSN did not exhibit any toxic effects to the panel of cells tested, the cytotoxicity of Nano-pPAAM was clearly directed towards the LAT-1 overexpressing cancer cells but not the normal cells in a dose-dependent manner. (FIG. 6D) Conversely, the exposure of these cell lines to cisplatin led to the indiscriminate killing of cancer and normal cell lines in a dose-dependent manner (FIG. 6D). Collectively, our results showed that Nano-pPAAM could selectively kill cancer cells by targeting the LAT-1 overexpressed cancer cells.

### Example 4

### Intracellular ROS Inducing Properties of Nano-pPAAM

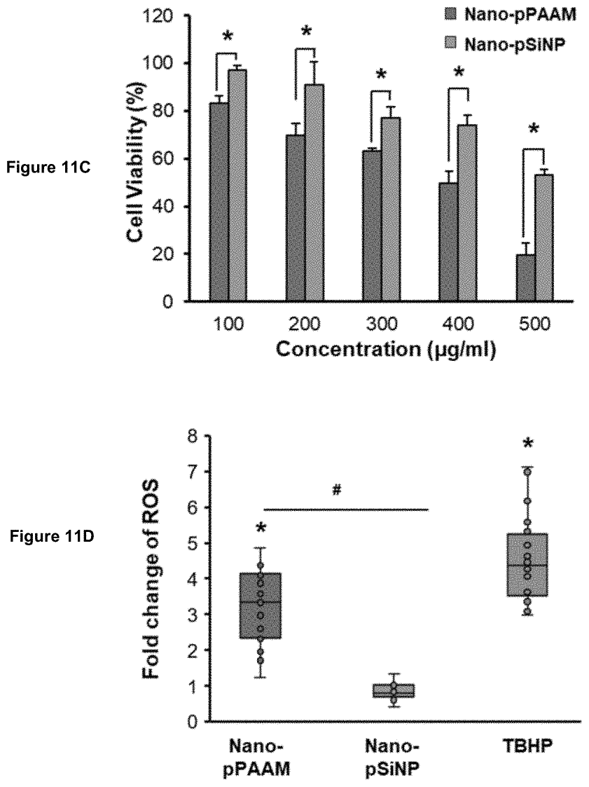

Previous studies have shown that silica NPs were able to stimulate the generation of intracellular ROS in several different mammalian cell lines (Yu, T. et al., ACS Nano 2011, 5 (7): 5717-28). Overproduction of ROS can lead to several effects such as peroxidation of lipids, DNA damage and consequently apoptosis (Blair, I. A., J Biol Chem 2008, 283(23): 15545-9]. Using the cell-permeant ROS sensitive CellROX dye, we observed that there was a slight increase in ROS level in the NH₂-MSN treated MDA-MB-231 cells relative to the untreated control group. In contrast, the intracellular ROS level was significantly higher in the Nano-pPAAM treated MDA-MB-231 (FIG. 11A). When the cells were treated with N-acteyl-L-cysteine (NAC, 4 mM), a potent ROS inhibitor, Nano-pPAAM mediated cytotoxicity in MDA-MB-231 cells was attenuated by ˜40% compared with the group without NAC treatment. This observation implies that Nano-pPAAM induced cell death is mediated via the elevated toxic level of intracellular ROS (FIG. 11B).

To gain further insights into the relationship between particles properties and ROS generating capability of Nano-pPAAM, we synthesized a size-matched non-porous variant of L-phenylalanine functionalized silica nanoparticles (Nano-pSiNP). The physicochemical traits of Nano-pSiNP are shown in Table 3 and FIG. 5. As shown in FIG. 11C, the removal of the mesoporous structure diminished the anticancer efficacy of Nano-pSiNP by as much as 30% compared with Nano-pPAAM. A reduction of pore-voids-associated surface area in the non-porous variant could lead to a decrease in the availability of the free silanol moieties (Si—OH) to generate intracellular reactive oxygen species (ROS) (Rabolli, V. et al., Nanotoxicology 2010, 4(3): 307-18). This view is in line with the widely accepted paradigm of NPs induced oxidative stress as the principal modus operandi to induce cellular toxicity (Fu, P. P. et al., J Food Drug Anal 2014, 22(1): 64-75). Consistent with this notion, when we measured the ROS inducing capability of both the Nano-pPAAM (porous) and Nano-pSiNP (non-porous), we noted that the intracellular ROS level is significantly higher (˜3.4 times) in the Nano-pPAAM group, yet we did not observe any significant changes to ROS level in the cells treated with the Nano-pSiNP variant relative to the untreated group (FIG. 11D). This observation reveals the critical role of particle porosity in governing the ROS-inducing capability and cancer-killing effect of Nano-pPAAM. This observation is also consistent with our previous study that showed the expression of intracellular ROS level was inversely correlated to the porosity of silica NPs (Tay, C. Y. et al., ACS Nano 2017, 11(3): 2764-2772).

### Example 5

### Dual Activation of Intrinsic and Extrinsic Apoptotic Pathways by Nano-pPAAM

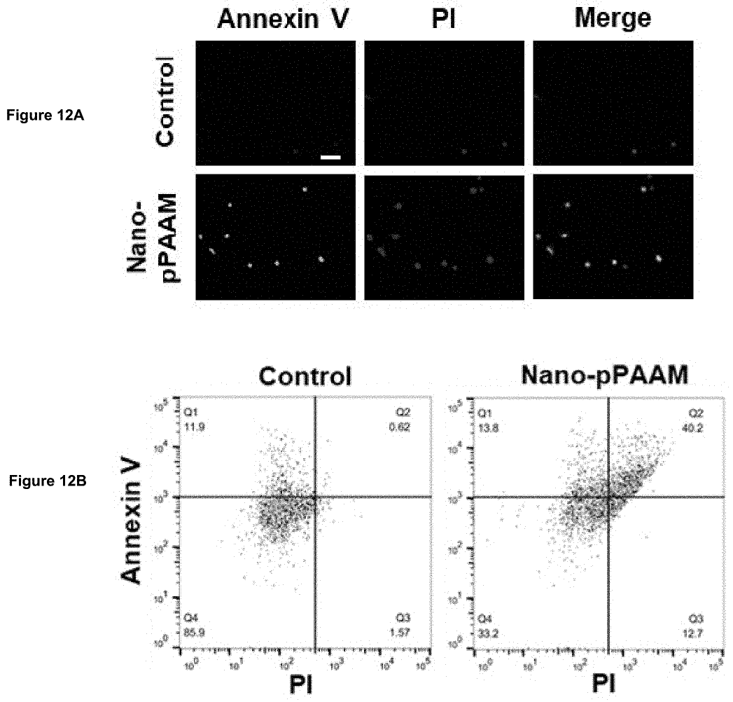

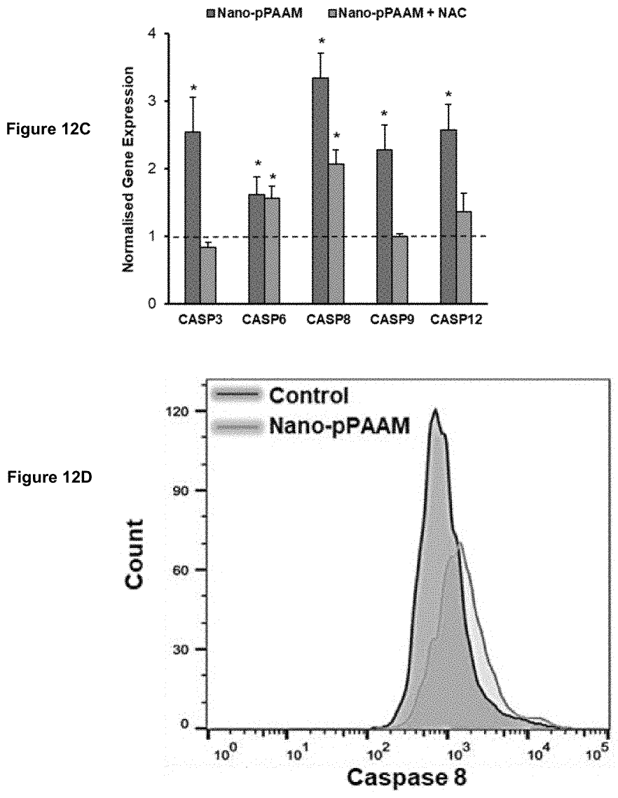

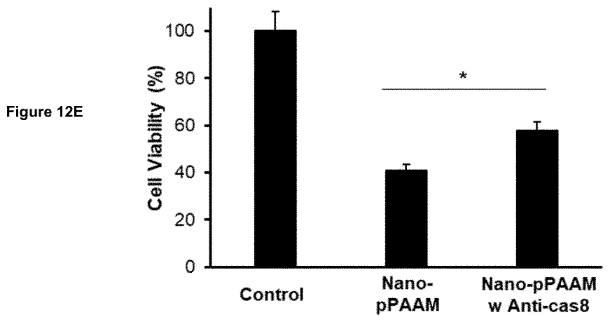

Although both particle size and porosity appear to be important determinants for the anti-cancer efficacy of Nano-pPAAM, the mechanistic cancer-killing action of Nano-pPAAM remains unclear. Generally, cells can die either by necrosis or apoptosis. Necrosis refers to accidental cell death resulting from cellular trauma with loss of plasma membrane integrity and rapid release of intracellular content. Conversely, apoptosis is a naturally occurring programmed cell death that is characterized by the process of autonomous cellular dismantling (Fink, S. L. and Cookson, B. T., Infect Immun 2005, 73(4): 1907-16). ROS has been implicated in both modes of cell death (Ryter, S. W. et al., Antioxid Redox Signal 2007, 9(1): 49-89). In the case of MDA-MB-231 cells treated with Nano-pPAAM (500 μg/ml), all of the cells were stained positive with both PI and Alexa Fluor 488 conjugated Annexin V (FIG. 12A), suggesting that apoptosis is the primary mode of cell death that is mediated by Nano-pPAAM. This notion was further supported by the flow cytometric analysis, which showed a significant increase in the number of PI and Annexin V double-positive cells (FIG. 12B).

We next investigated the apoptotic pathways invoked by Nano-pPAAM, as cellular apoptosis can occur via the intrinsic or extrinsic caspases-regulated molecular pathways. The intrinsic pathway is activated by intracellular stress signals, while the extrinsic pathway is triggered by the coupling of extracellular death ligands to the cell-surface death receptors (Elmore, S., Toxicol Pathol 2007, 35(4): 495-516). Initiation of the apoptotic machinery requires the sequential activation of the apoptotic initiator caspases (e.g. caspase-8, -9, -12) and effector caspases (e.g. caspase-3, -6) (Andreau, K. et al., Biochem Res Int 2012, 2012: 493894; Li, J. and Yuan, J., Oncogene 2008, 27(48): 6194-206). Consistent with the apoptotic assay results, our real time quantitative PCR analysis revealed that the mRNA levels of caspase-3, -6, -8, -9 and -12 were significantly upregulated as early as 8 h post Nano-pPAAM treatment (FIG. 12C). The pre-treatment of cells with NAC followed by Nano-pPAAM repressed the mRNA of caspase-3, -9, and -12 to a level that was comparable to the untreated control cells. In contrast, the mRNA level of caspase-8 remained elevated compared with untreated control even in the NAC treated group. Caspase-8 is a cystein protease that is known to play a unique role in the extrinsic apoptotic signalling pathway via the death receptors such as apoptosis antigen 1 (FAS) and TNF-related apoptosis-inducing ligand (Elmore, S., Toxicol Pathol 2007, 35(4): 495-516; Juo, P. et al., Current Biology 1998, 8(18): 1001-1008; Sprick, M. R., et al., Immunity 2000, 12(6): 599-609]. The activation of caspase-8 by Nano-pPAAM treatment was further confirmed using flow cytometry (FIG. 12D). Additionally, we noted that pharmacological inhibition of caspase-8 by z-IETD-FMK (5 μM) led to a partial (˜18%) attenuation of Nano-pPAAM induced apoptosis (FIG. 12E), suggesting that the activation of caspase-8 is only partly involved in the Nano-pPAAM induced apoptosis. Nevertheless, our data suggests the synergistic ROS-dependent and-independent apoptotic signalling as a potential anti-cancer mode-of-action of Nano-pPAAM.

### Example 6

### In Vitro and In Vivo Antitumoral Properties of Nano-pPAAM

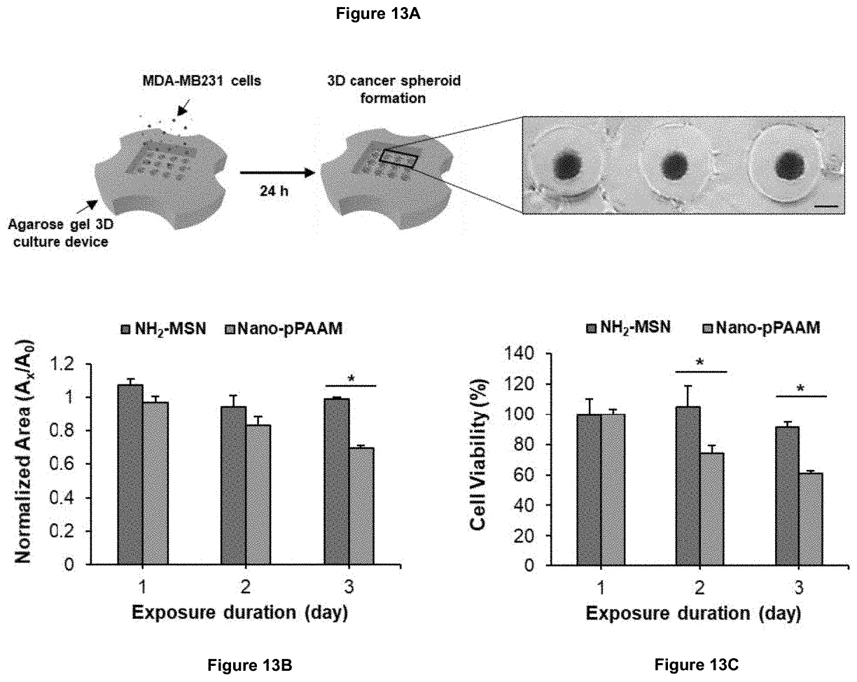

To better evaluate the anticancer effects of Nano-pPAAM in a more tumor-like setting, we established a micropatterned hydrogel platform to generate uniformly sized 3-dimensional MDA-MB-231 tumor spheroids (FIG. 13A) (Chia, S. L. et al., Small 2015, 11(6): 702-12). The patterned breast tumor spheroid has an average diameter of ˜120 μm (FIG. 13A) and was treated with or without Nano-pPAAM (500 μg/ml). Consistent with our 2D culture in vitro data, NH₂-MSN treatment did not induce any significant changes to the size nor viability to the cancer spheroids relative to the untreated control. Conversely, we observed a steady concomitant time-dependent decrease in spheroid size and cell viability in the Nano-pPAAM treated samples (FIG. 13B and C). A significant reduction in cell viability in the 3D tumor spheroid model was only observed 2 days post Nano-pPAAM treatment, whereas there was a ˜80% kill rate in the 2D MDA-MB-231 monolayer model at an equivalent concentration of Nano-pPAAM within 1 day (FIG. 2D). This difference is expected as the dense 3D packing of the cancer cells, and tumor ECM are formidable barriers that can impede the penetration of nanoparticles into the tumor (Tay, C. Y. et al., Advanced Functional Materials 2016, 26(23): 4046-4065). Nevertheless, we were still able to achieve ˜40% decrease in cell viability and ˜38% suppression of tumor growth in the Nano-pPAAM treated 3D tumor spheroids group. Our results demonstrate the utility of Nano-pPAAM to exert its anti-cancer properties, even in a complex 3D microenvironment.

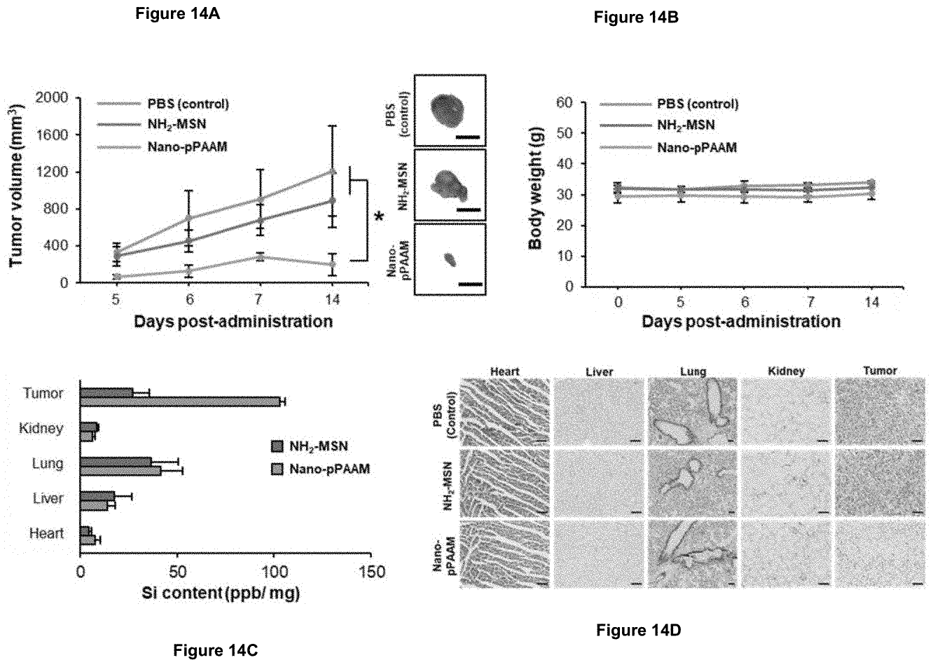

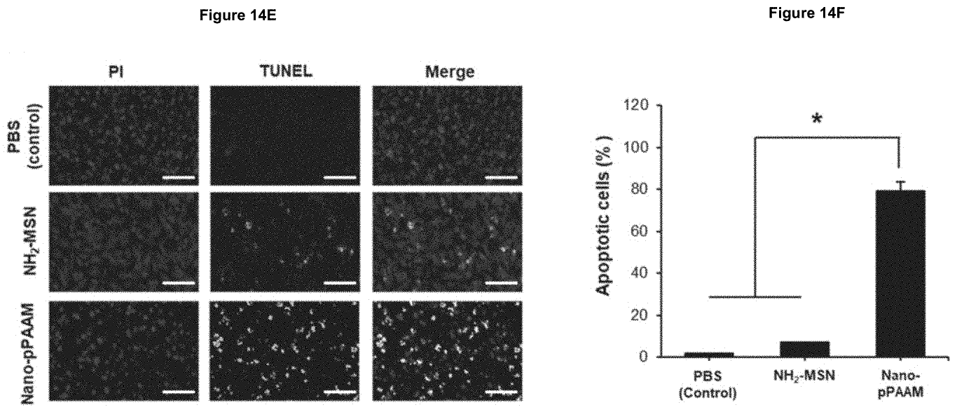

The antitumor efficacy of Nano-pPAAM was further evaluated using a human breast cancer xenograft-bearing NOD scid gamma mouse (NSG) mice model. MDA-MB-231 cells were subcutaneously implanted into the dorsal flanks of the mice (n=5). Mice were randomly divided into three groups, namely, control group (PBS), NH₂-MSN and Nano-pPAAM. As shown in FIG. 14A, the intra-tumor injection of the NH₂-MSN (3000 μg/ml in PBS, 100 μl) did not inhibit tumor growth, which is consistent with our in vitro results (FIG. 2D and 13B, C). By day 14, similar tumor volumes (i.e., 600-1200 mm³) were recorded in both the NH₂-MSN treatment and PBS control groups. In contrast, tumor growth was well inhibited in mice treated with the Nano-pPAAM, limiting the tumor volume to <300 mm³. Comparison of the excised tumors at the end of the 14 days experimental period revealed that tumor growth was noticeably reduced in the Nano-pPAAM treatment group. We also found that during the entire experiment, the mouse body weights did not exhibit any significant difference in all the three experimental groups (FIG. 14B). In terms of the biodistribution of the nanoparticles, ICP-MS analysis of the target organs revealed the majority of the intratumorally administered nanoparticles were retained in the tumor, while traces of Si content could be detected in the kidney, lung, liver and heart (FIG. 14C). Despite the presence of Si, hematoxylin and eosin (H&E) staining did not reveal any damage nor abnormalities in the respective tissues (FIG. 14D). However, specific to the Nano-pPAAM treated group, severe tumor damage as indicated by the extensive coverage of vacuoles, condensed nuclei and altered cell morphology was observed. This observation is in line with the drastic reduction in tumor volume as a result of the Nano-pPAAM treatment (FIG. 14A). In addition to the H&E analysis, the excised tumors sliced were also stained for apoptosis DNA fragmentation using the TUNEL assay (FIG. 14E). Interestingly, visual inspection of the immunostaining images revealed that the number of TUNEL positive cells is by far the greatest in the Nano-pPAAM samples. While the number of apoptotic cells were marginal (<10%) in both the control and NH₂-MSN treated samples, we observed significant apoptosis (˜80%) in the Nano-pPAAM treated xenograft tumor (FIG. 14F). This observation corroborates our earlier findings (FIG. 12A), suggesting that the primary mode of Nano-pPPAM anti-cancer efficacy is via induction of apoptosis. Collectively, these data suggest that Nano-pPAAM is highly efficacious as a novel antitumoral nano-agent.

### Example 7

### Anti-Cancer Efficacy of Nano-pPAAM Compared to L-phenylalanine

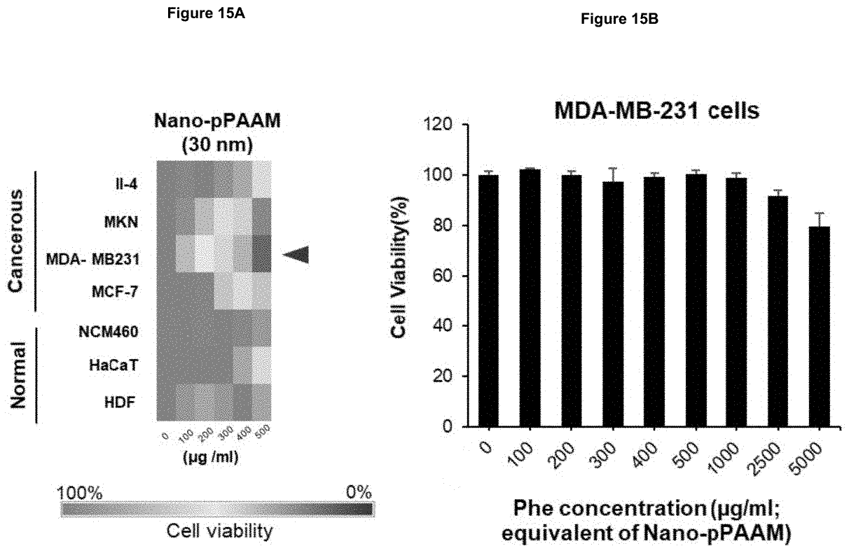

The efficacy of Nano-pPAAM to kill cancer cells was compared to that of a L-phenylalanine solution (corrected to equivalent concentration of Nano-pPAAM) in MDA-MB-231 human breast cancer cells. L-phenylalanine treatment did not result in any significant decrease in cancer cell viability even at the extreme concentration of 1000 μg/ml equivalent concentration of Nano-pPAAM (FIG. 15). In contrast, 500 μg/ml of Nano-pPPAM achieved >80% cell death. Therefore, it can be concluded that L-phenylalanine does not contribute to the observed cancer killing effect across the concentrations of Nano-pPAAM that were found to be potent against MDA-MB-231 cells.

This result, coupled with the weak anti-cancer effect of NHs-MSN (unconjugated control), strongly suggest that the anti-cancer property of Nano-pPAAM is a result of the strong synergism between the mesoporous silica core and L-phenylalanine “cloak”.

### SUMMARY

A new class of drug-free nano-scale porous amino acid mimic with intrinsic anti-cancer properties has been developed. The working principle of Nano-PAAM is to exploit the amino acid metabolic vulnerabilities of cancer cells and the ROS inducing capability of the porous MSN core to deliver a deadly level of oxidative stress to the cancer cells. Among the panel of Nano-PAAM, Nano-pPAAM was revealed as the prime candidate with superior therapeutic efficacy and cancer-selectivity towards a panel of different cancer cell lines. We further delineated the important physicochemical parameters and the apoptosis pathways that are critical to the therapeutic action of Nano-pPAAM. We also demonstrated that Nano-pPAAM treatment can reduce tumor growth by ˜60% in an MDA-MB 231 xenograft in vivo mice model, underscoring the potential clinical utility of Nano-pPAAM. Our results clearly show that NPs can be armed with several intrinsic anti-cancer features that could either be exploited as a stand-alone or adjuvant novel antitumor agent.

To the best of our knowledge, our invention is the first of its kind, to use amino-acid conjugated mesoporous silica nanoparticles to exert a potent anti-cancer effect and achieve selective killing of cancer cells while sparing the healthy normal cells in 2D and 3D in vitro models.

### BIBLIOGRAPHY

Ahmed A. Moosa, B. F. S., American Journal of Materials Science 2017, 7 (6), 223-231.

Andreau, K.; Leroux, M.; Bouharrour, A., Biochem Res Int 2012, 2012, 493894.

Bartczak, D.; Kanaras, A. G., Langmuir 2011, 27 (16), 10119-23.

Barua, S.; Mitragotri, S., Nano Today 2014, 9 (2), 223-243.

Blair, I. A., J Biol Chem 2008, 283 (23), 15545-9.

Chia, S. L.; Tay, C. Y.; Setyawati, M. I.; Leong, D. T., Small 2015, 11 (6), 702-12.

Deber, C. M.; Stone, T. A., Peptide Science 2019, 111 (1).

De Jong, W. H.; Borm, P. J., Int J Nanomedicine 2008, 3 (2), 133-49.

Elmore, S., Toxicol Pathol 2007, 35 (4), 495-516.

Fink, S. L.; Cookson, B. T., Infect Immun 2005, 73 (4), 1907-16.

Fotiadis, D.; Kanai, Y.; Palacin, M., Mol Aspects Med 2013, 34 (2-3), 139-58.

Fu, P. P.; Xia, Q.; Hwang, H. M.; Ray, P. C.; Yu, H., J Food Drug Anal 2014, 22 (1), 64-75.

Geier, E. G.; Schlessinger, A.; Fan, H.; Gable, J. E.; Irwin, J. J.; Sali, A.; Giacomini, K. M., Proc Natl Acad Sci USA 2013, 110 (14), 5480-5.

Guo, H.; Qian, H.; Sun, S.; Sun, D.; Yin, H.; Cai, X.; Liu, Z.; Wu, J.; Jiang, T.; Liu, X., Chem Cent J 2011, 5(1), 1.

Hafliger, P.; Charles, R. P., Int J Mol Sci 2019, 20 (10): 2428.

Hayase, S.; Kumamoto, K.; Saito, K.; Kofunato, Y.; Sato, Y.; Okayama, H.; Miyamoto, K.; Ohki, S.; Takenoshita, S., Oncol Lett 2017, 14 (6), 7410-7416.

Hare, J. I.; Lammers, T.; Ashford, M. B.; Puri, S.; Storm, G.; Barry, S. T., Challenges and strategies in anti-cancer nanomedicine development: An industry perspective. Adv Drug Deliv Rev 2017, 108, 25-38.

Hao, N.; Jayawardana, K. W.; Chen, X.; Yan, M., ACS Appl Mater Interfaces 2015, 7 (2), 1040-5.

He, Q.; Shi, J., Journal of Materials Chemistry 2011, 21 (16).

Hua, S.; de Matos, M. B. C.; Metselaar, J. M.; Storm, G., Front Pharmacol 2018, 9, 790.

Huang, C.; Zhang, Y.; Yuan, H.; Gao, H.; Zhang, S., Nano Lett 2013, 13 (9), 4546-50.

Huang, Y. B.; Wang, X. F.; Wang, H. Y.; Liu, Y.; Chen, Y., Mol Cancer Ther 2011, 10 (3), 416-26.

Huang, Z.; Xu, H.; Meyers, A. D.; Musani, A. I.; Wang, L.; Tagg, R.; Barqawi, A. B.; Chen, Y. K., Technol Cancer Res Treat 2008, 7 (4), 309-20.

Juo, P.; Kuo, C. J.; Yuan, J.; Blenis, J., Current Biology 1998, 8 (18), 1001-1008.

Kim, S. E.; Zhang, L.; Ma, K.; Riegman, M.; Chen, F.; Ingold, I.; Conrad, M.; Turker, M. Z.; Gao, M.; Jiang, X.; Monette, S.; Pauliah, M.; Gonen, M.; Zanzonico, P.; Quinn, T.; Wiesner, U.; Bradbury, M. S.; Overholtzer, M., Nat Nanotechnol 2016, 11 (11), 977-985.

Lehman, S. E.; Morris, A. S.; Mueller, P. S.; Salem, A. K.; Grassian, V. H.; Larsen, S. C., Environ Sci Nano 2016, 3 (1), 56-66.

Li, J.; Yuan, J., Oncogene 2008, 27 (48), 6194-206.

Li, L.; Di, X.; Wu, M.; Sun, Z.; Zhong, L.; Wang, Y.; Fu, Q.; Kan, Q.; Sun, J.; He, Z., Nanomedicine 2017, 13 (3), 987-998.

Li, S. D.; Huang, L., Biochim Biophys Acta 2009, 1788 (10), 2259-66.

Liechty, W. B.; Kryscio, D. R.; Slaughter, B. V.; Peppas, N. A., Annu Rev Chem Biomol Eng 2010, 1, 149-73.

Lopez-Lazaro, M., Oncoscience 2015, 2 (10), 857-66.

Luo, Z.; Xu, Y.; Ye, E.; Li, Z.; Wu, Y. L., Macromol Rapid Commun 2019, 40 (5), e1800029.

Maddocks, O. D. K.; Athineos, D.; Cheung, E. C.; Lee, P.; Zhang, T.; van den Broek, N. J. F.; Mackay, G. M.; Labuschagne, C. F.; Gay, D.; Kruiswijk, F.; Blagih, J.; Vincent, D. F.; Campbell, K. J.; Ceteci, F.; Sansom, O. J.; Blyth, K.; Vousden, K. H., Nature 2017, 544 (7650), 372-376.

Minchinton, A. I.; Tannock, I. F., Nat Rev Cancer 2006, 6 (8), 583-92.

Pandele, A. M.; Andronescu, C.; Ghebaur, A.; Garea, S. A.; lovu, H., Materials (Basel) 2018, 12 (1).

Patra, J. K.; Das, G.; Fraceto, L. F.; Campos, E. V. R.; Rodriguez-Torres, M. D. P.; Acosta-Torres, L. S.; Diaz-Torres, L. A.; Grillo, R.; Swamy, M. K.; Sharma, S.; Habtemariam, S.; Shin, H. S., J Nanobiotechnology 2018, 16 (1), 71.

Peña-Alonso, R.; Rubio, F.; Rubio, J.; Oteo, J. L., Journal of Materials Science 2006, 42 (2), 595-603.

Rabolli, V.; Thomassen, L. C.; Princen, C.; Napierska, D.; Gonzalez, L.; Kirsch-Volders, M.; Hoet, P. H.; Huaux, F.; Kirschhock, C. E.; Martens, J. A.; Lison, D., Nanotoxicology 2010, 4 (3), 307-18.

Reisch, A.; Heimburger, D.; Ernst, P.; Runser, A.; Didier, P.; Dujardin, D.; Klymchenko, A. S., Advanced Functional Materials 2018, 28 (48).

Ryter, S. W.; Kim, H. P.; Hoetzel, A.; Park, J. W.; Nakahira, K.; Wang, X.; Choi, A. M., Antioxid Redox Signal 2007, 9 (1), 49-89.

Saftig, P.; Klumperman, J., Nat Rev Mol Cell Biol 2009, 10 (9), 623-35.

Saito, Y.; Li, L.; Coyaud, E.; Luna, A.; Sander, C.; Raught, B.; Asara, J. M.; Brown, M.; Muthuswamy, S. K., Nature 2019, 569 (7755), 275-279.

Scalise, M.; Pochini, L.; Galluccio, M.; Indiveri, C., Biochim Biophys Acta 2016, 1857 (8), 1147-1157.

Senapati, S.; Mahanta, A. K.; Kumar, S.; Maiti, P., Signal Transduct Target Ther 2018, 3, 7.

Sercombe, L.; Veerati, T.; Moheimani, F.; Wu, S. Y.; Sood, A. K.; Hua, S., Front Pharmacol 2015, 6, 286.

Setyawati, M. I.; Tay, C. Y.; Leong, D. T., Effect of zinc oxide nanomaterials-induced oxidative stress on the p53 pathway. Biomaterials 2013, 34 (38), 10133-42.

Sprick, M. R.; Weigand, M. A.; Rieser, E.; Rauch, C. T.; Juo, P.; Blenis, J.; Krammer, P. H.; Walczak, H., Immunity 2000, 12 (6), 599-609.

Su, H.; Wang, Y.; Liu, S.; Wang, Y.; Liu, Q.; Liu, G.; Chen, Q., Acta Pharm Sin B 2019, 9 (1), 49-58.

Tauqir, N. A., Journal of Environmental & Agricultural Sciences 2016, 9, 14.

Tay, C. Y.; Cai, P. Q.; Setyawati, M. I.; Fang, W. R.; Tan, L. P.; Hong, C. H. L.; Chen, X. D.; Leong, D. T., Nanoparticles Strengthen Intracellular Tension and Retard Cellular Migration. Nano Lett 2014, 14 (1), 83-88.

Tay, C. Y.; Leong, D. T., Inorganic nanoparticles as tubulin binding agents for cancer therapy. Nanomedicine-Uk 2014, 9 (14), 2075-2077.

Tay, C. Y.; Muthu, M. S.; Chia, S. L.; Nguyen, K. T.; Feng, S.-S.; Leong, D. T., Advanced Functional Materials 2016, 26 (23), 4046-4065.

Tay, C. Y.; Setyawati, M. I.; Leong, D. T., ACS Nano 2017, 11 (3), 2764-2772.

Tay, C. Y.; Setyawati, M. I.; Xie, J. P.; Parak, W. J.; Leong, D. T., Back to Basics: Exploiting the Innate Physico-chemical Characteristics of Nanomaterials for Biomedical Applications. Adv Funct Mater 2014, 24 (38), 5936-5955.

Tsun, Z. Y.; Possemato, R., Semin Cell Dev Biol 2015, 43, 22-32.

Vucetic, M.; Cormerais, Y.; Parks, S. K.; Pouyssegur, J., Front Oncol 2017, 7, 319.

Wu, Z.; Yang, H.; Archana, G.; Rakshit, M.; Ng, K. W.; Tay, C. Y., Nanotoxicology 2018, 12 (10), 1215-1229.

Yang, Y.-W., MedChemComm 2011, 2 (11).

Yu, T.; Malugin, A.; Ghandehari, H., ACS Nano 2011, 5 (7), 5717-28.

Zhang, S.; Li, J.; Lykotrafitis, G.; Bao, G.; Suresh, S., Adv Mater 2009, 21, 419-424.

Zhu, C.; Gao, Y.; Li, H.; Meng, S.; Li, L.; Francisco, J. S.; Zeng, X. C., Proc Natl Acad Sci U S A 2016, 113 (46), 12946-12951.

What is claimed is:

1. An amino acid-functionalized nanoparticle, comprising:
i) a nanoparticle having a dimension in the range of units of nanometers to 100 nanometers, and having an external surface; and
ii) a plurality of amino acid molecules conjugated to the external surface of the nanoparticle, the amino acid molecules having a chemical property that can induce a cancer cell to ingest the functionalized nanoparticle;
wherein said nanoparticle is a mesoporous silica nanoparticle and/or other ROS-generating nanoparticle; and
wherein said amino acid molecules are selected from the group consisting of histidine, isoleucine, leucine, lysine, methionine, phenylalanine, threonine, tryptophan, and valine.

2. The amino acid-functionalized nanoparticle of claim 1, wherein the conjugated amino acid is L-phenylalanine.

3. The amino acid-functionalized nanoparticle of claim 1, wherein the nanoparticle has a dimension in the range of 10-150 nm, preferably about 30 nm.

4. The amino acid-functionalized nanoparticle of claim 1, wherein the nanoparticle is a mesoporous silica nanoparticle and has mesopore structures in the range of about 1 nm to about 5 nm in size.

5. The amino acid-functionalized nanoparticle of claim 1, wherein the biological effect is ROS-induced cancer cell apoptosis.

6. The amino acid-functionalized nanoparticle of claim 1, wherein the cancer cell overexpresses an L-type amino acid transporter 1 (LAT1) compared to a normal cell.

7. The amino acid-functionalized nanoparticle of claim 1, wherein the cancer cell is selected from the group comprising breast cancer, gastric cancer and skin cancer.

8. A method of production of an amino acid-functionalized nanoparticle of claim 1, comprising:
a) Mix and dissolve an essential amino acid, ethyl(dimethylaminopropyl) carbodiimide (EDC), and N-Hydroxy succinimide (NHS) in PBS buffer;
b) Form a suspension of NH₂-functionalized mesoporous silicon nanoparticles or other NH₂-functionalized ROS-generating nanoparticles in PBS buffer;
c) Mix the suspension from b) with the solution from a) at room temperature; and
d) Extract the final product.

9. The method of claim 8, further comprising freeze-drying the amino acid-functionalized nanoparticles product for storage.

10. The method of claim 8, wherein the nanoparticle has a dimension in the range of 10-80 nm, preferably about 30 nm.

11. The method of claim 8, wherein the final product is an amino acid-functionalized mesoporous silica nanoparticle.

12. The method of claim 11, wherein the mesoporous silica nanoparticle has mesopore structures in the range of about 2 nm to about 3 nm in size.

13. The method of claim 8, wherein the essential amino acid is selected from the group consisting of Trp, Ile, Met and Phe.

14. The method of claim 8, wherein the essential amino acid is L-phenylalanine.

15. A pharmaceutical composition comprising at least one amino acid-functionalized nanoparticle of claim 1 and an acceptable pharmaceutical vehicle for the treatment of cancer in a subject.

16. The pharmaceutical composition of claim 15, wherein the cancer comprises cancer cells that overexpress an L-type amino acid transporter 1 (LAT1) compared to a normal cell.

17. The pharmaceutical composition of claim 15, wherein the cancer is selected from the group consisting of breast cancer, gastric cancer and skin cancer.

18. A method of treatment comprising administering to a subject in need of such treatment an effective amount of an amino acid-functionalized nanoparticle of claim 1.

19. The method of claim 18, wherein the subject has cancer.

20. The method of claim 19, wherein the cancer comprises cancer cells that overexpress an L-type amino acid transporter 1 (LAT1) compared to a normal cell.

21. The method of claim 19, wherein the cancer is selected from the group consisting of breast cancer, gastric cancer and skin cancer.
